(19) United States
(12) Patent Application Publication
(10) Pub. No.: US 2006/0148893 A1
(43) Pub. Date: Jul. 6, 2006

(54) Chemical compounds

(76) Inventors: Jean-Baptiste E. Blanc, Durham, NC (US); Rodolfo Cadilla, Durham, NC (US); David John Cowan, Durham, NC (US); Istvan Kaldor, Durham, NC (US); Andrew L. Larkin, Durham, NC (US); Eugene Lee Stewart, Durham, NC (US); Ryan Paul Trump, Durham, NC (US); Philip Stewart Turnbull, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/560,012

(22) PCT Filed: Jun. 9, 2004
(86) PCT No.: PCT/US04/18252

§ 371 Date: Dec. 8, 2005

(30) Foreign Application Priority Data
Jun. 10, 2003 (US) 60477252

(51) Int. Cl. A61K 31/277; C07C 255/49

(52) U.S. Cl. 514/521; 558/408

(57) ABSTRACT

This invention relates to non-steroidal compounds that are or are believed to be modulators of androgen, glucocorticoid, mineralocorticoid, and progesterone receptors, and also to the methods for the making and use of such compounds.

### FIELD OF THE INVENTION

This invention relates to non-steroidal compounds that are or are believed to be modulators of androgen, glucocorticoid, mineralocorticoid, and progesterone receptors, and also to the methods for the making and use of such compounds.

### BACKGROUND OF THE INVENTION

Nuclear receptors are a class of structurally related gene expression modulators that act as ligand-dependent transcription factors (R. M. Evans, Science 240, 889 (1988)). The steroid receptors, namely the androgen receptor, the estrogen receptor, the glucocorticoid receptor, the mineralocorticoid receptor, and the progesterone receptor represent a subclass of the nuclear receptor superfamily. Nuclear receptor ligands in this subclass exert their effects by binding to an intracellular steroid hormone receptor. After the receptor-ligand complex is translocated to the nucleus of the cell, the complex binds to recognition sites on DNA, which allows for the modulation of certain genes.

Certain substances have demonstrated the ability to exhibit their activity in a tissue selective manner. In other words, tissue selectivity allows a nuclear receptor ligand to function as an agonist in some tissues, while having no effect or even an antagonist effect in other tissues. The term “selective receptor modulator” (SRM) has been given to these molecules. A synthetic compound that binds to an intracellular receptor and mimics the effects of the native hormone is referred to as an agonist. A compound that inhibits the effect of the native hormone is called an antagonist. The term “modulators” refers to compounds that have a spectrum of activities ranging from full agonism to partial agonism to full antagonism. The molecular basis for this tissue selective activity is not completely understood. Without being limited to any particular explanation, particular ligands put nuclear receptors in different conformational states. These states dictate the ability of coactivators, corepressors, and other proteins to be recruited by the nuclear receptor (“NR”). The unique cofactor-NR ensembles are the gene transcription factors that are thought to modulate tissue selective effects.

Ligand-mediated effects through the action of nuclear receptors are not limited to the classical genotropic mechanism outlined above. It is thought that some, if not all, of the separation of anabolic and general homeostatic effects from the stimulation of sexual tissues can be explained by a particular ligand's ability to potentiate non-genotropic pathways. One example of liganded nuclear receptor induction of non-genotropic pathways is found in the work of S. C. Manolagas et al., Cell, 104, 719-730. The action of a sex steroid NR on osteoblasts and other cell types is shown to involve the Src/Shc/ERK signaling pathway. This activity is mediated through the ligand binding domain of the sex steroid nuclear receptor alone. The NR DNA-binding domain is not required to attenuate etoposide-induced apoptosis in HeLa cells. An NR lacking the DNA binding domain cannot function in the classical mode, acting as a transcription factor.

Nuclear receptor steroid ligands are known to play important roles in the health of both men and women. In regard to men's health, testosterone (T) and dihydrotestosterone (DHT), for example, are endogenous steroidal ligands for the androgen receptor that likely play a role in every tissue type found in the mammalian body. During the development of the fetus, androgens play a role in sexual differentiation and development of male sexual organs. Further sexual development is mediated by androgens during puberty. Androgens play diverse roles in the adult including stimulation and maintenance of male sexual accessory organs and maintenance of the musculoskeletal system. Cognitive function, sexuality, aggression, and mood are some of the behavioral aspects mediated by androgens. Androgens affect the skin, bone, and skeletal muscle, as well as blood lipids and blood cells.

The study of androgen action and male reproductive dysfunction continues to expand significantly. In fact, only recently has the definition of a disease state been associated with hormonal changes that occur in aging men. This syndrome, previously referred to as Andropause, has more recently been described as Androgen Deficiency in the Aging Male, or “ADAM” (A. Morales and J. L. Tenover, Urologic Clinics of North America (2002 Nov.) 29(4) 975.) The onset of ADAM is unpredictable and its manifestations are subtle and variable. Clinical manifestations of ADAM include fatigue, depression, decreased libido, erectile dysfunction as well as changes in cognition and mood.

Published information indicates that androgen replacement therapy (ART) in men may have benefits in terms of improving body composition parameters (e.g. bone mineral density, increasing muscle mass, and strength) as well as improving libido and mood in some men. Therefore, andrologists and other specialists are increasingly using ART for the treatment of the symptoms of ADAM—though there is due caution given androgen's, like testosterone, potential side effects. Nonetheless, there is increasing scientific rational of and evidence for androgen deficiency and treatment in the aging male. Current testosterone-based ART therapies include injections, skin patches, gel-based formulations, and oral preparations. All of these therapies are somewhat efficacious in the treatment of ADAM, but, due to the dramatic fluctuations in plasma T-levels following treatment, success with these therapies has been variable.

Testosterone replacement products, such as AndroGel® (1% testosterone gel CIII, marketed by Solvay Pharmaceuticals) are emerging as a treatment of choice among physicians. Such products, however, fail to correctly mimic physiological testosterone levels and have potential side effects including exacerbation of pre- existing sleep apnoea, polycythemia, and/or gynaecomastia. Furthermore, the longer-term side effects on target organs such as the prostate or the cardiovascular system are yet to be fully elucidated. Importantly, the potential carcinogenic effects of testosterone on the prostate prevent many physicians from prescribing it to older men (i.e. age>60 years) who, ironically, stand to benefit most from treatment. Also, all of the existing treatment options have fundamental problems with their delivery mechanism. The need for a novel selective androgen receptor modulator (SARM) is obviated by the potential side effect profile manifested in conventional treatments. A SARM would ideally have all the beneficial effects of endogenous androgens, while sparing sexual accessory organs, specifically the prostate.

In regard to female health, progesterone, the endogenous ligand for the progesterone receptor (“PR”), plays an important role in female reproduction during the various stages of the ovarian cycle and during pregnancy. Among other things, progesterone prepares the endometrium for implantation, regulates the implantation process, and helps maintain pregnancy. The therapeutic use of synthetic versions of progesterone (progestins) stems from progesterone's ability to regulate endometrial proliferation. In fact, progestins are included as part of hormone replacement therapy (“HRT”) in women to reduce the incidence of endometriosis. Unfortunately, the effectiveness of therapy is tempered by undesired side-effect profiles. Chronic progestin therapy or continuous estrogen replacement regimens are often associated with increased bleeding. Excessive stimulatory effects on the endometrial vasculature may result in proliferation and fragility.

Compounds that modulate the effects of progesterone binding to PR are believed useful in the treatment and/or prophylaxis of endometriosis and uterine fibroid processes. Progesterone receptor antagonists such as mifepristone, also known as RU486, and other PR modulators can inhibit endometrial proliferation at high estradiol concentrations in primates. Human clinical data with mifepristone supports the efficacy of a PR antagonist in endometriosis (D. R. Grow et. al., J. Clin. Endocrin. Metab. 1996, 81). Despite enthusiasm for its use, RU-486 also acts as a potent ligand for the glucocorticoid receptor (“GR”). This cross-reactivity with the GR is associated with homeostatic imbalances.

Thus, modulators of nuclear steroid hormones that are highly specific for one receptor could offer greater benefit with less side effects in the treatment of both female and male related hormone responsive diseases.

### SUMMARY OF INVENTION

The present invention includes compounds of formula (I): 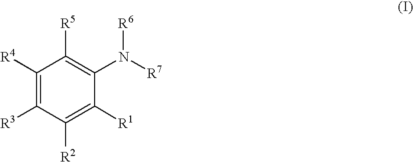
including salts, solvates, and physiologically functional derivatives thereof, wherein
R¹ is H, cyano, nitro, halogen, haloalkyl, alkyl, alkenyl, alkynyl, hydroxy, alkoxy, haloalkoxy, —OC(O)R⁸, or aryl;
R² is H, cyano, nitro, halogen, haloalkyl, alkyl, alkenyl, alkynyl, hydroxy, alkoxy, haloalkoxy, —OC(O)R⁸, or aryl;
R³ is cyano, nitro, halogen, haloalkyl, heterocyclyl, hydroxy, alkoxy, haloalkoxy, —OC(O)R⁸, —C(O)₂R⁸, —CONHR⁸, —C(O)R⁸, —S(O)ₙR⁸, —SO₂N(R⁸)₂, —NHC(O)R⁸, or —NHSO₂R⁸;
R⁴ is H, cyano, nitro, halogen, haloalkyl, alkyl, alkenyl, alkynyl, hydroxy, alkoxy, haloalkoxy, —OC(O)R⁸, or aryl;
R⁵ is H, cyano, nitro, halogen, haloalkyl, alkyl, alkenyl, alkynyl, hydroxy, alkoxy, haloalkoxy, —OC(O)R⁸, or aryl;
where at least one of R¹, R², R⁴, and R⁵ is not H;
each of R⁶ and R⁷ independently are selected from H or —(R^{a})ₓ-R⁹;
R⁸ is a C₁-C₈ alkylene chain, where x is 0 or 1;
each R⁸ independently is H, alkyl, alkenyl, alkynyl, cycloalkyl, heterocyclyl, aryl, aralkyl, heteroaryl, or heteroaralkyl; R⁹ is alkyl, haloalkyl, hydroxyalkyl, alkenyl, alkynyl, alkoxy, alkylthio, haloalkoxy, cycloalkyl, formyl, azido, or —NR¹⁰R¹¹;
R¹⁰and R¹¹ each independently are H, alkyl, —C(O)H, —C(O)R¹², —C(O)OR¹², or —SO₂R¹²; and
R¹² is alkyl.

In one embodiment R¹, R⁵, or both are H. In one embodiment R², R⁴, or both are H.

In one embodiment alkyl is C₁-C₈ alkyl, preferably alkyl is C₁-C₃ alkyl. In one embodiment alkoxy is C₁-C₆ alkoxy, preferably C₁-C₂ alkoxy. In one embodiment haloalkyl is C₁-C₆ haloalkyl, preferably haloalkyl is trifluoromethyl or trifluoroethyl. In one embodiment alkenyl is C₂-C₆ alkenyl, preferably alkenyl is isopropenyl, isobutenyl or allyl. In one embodiment, alkynyl is C₂-C₆, preferably alkynyl is propynyl. In one embodiment cycloalkyl is C₃-C₆ cycloalkyl, preferably cycloalkyl is cyclopropyl, cyclopentyl, or cyclohexyl.

In one embodiment R¹ or R⁵ is nitro, alkyl, haloalkyl, or halogen.

In one embodiment R² or R⁴ is nitro, cyano, alkyl, haloalkyl, halogen, or hydroxy.

In one embodiment R³ is cyano, nitro, or halogen. Preferably, one or more of R¹ , R², R⁴, or R⁵ is haloalkyl. More preferably R² or R⁴ are haloalkyl.

In one embodiment R³ is cyano, nitro, or halogen. Preferably, one or more of R¹, R², R⁴, or R⁵ is halogen. More preferably R² or R⁴ are halogen. Still more preferably R² or R⁴ is chloro.

Alternatively, R³is cyano and one or more of R¹, R², R⁴, or R⁵is nitro. Further alternatively, R³ is nitro and one or more of R¹, R², R⁴, or R⁵ is cyano. Still further, R³ is cyano and one of R² or R⁴ is cyano.

In one embodiment R^{a} is a substituted alkylene, substituted with one or more of alkyl, alkenyl, or hydroxyl.

Particularly preferred compounds of the present invention include:
4-[(Cyclopropylmethyl)(propyl)amino]-2-(trifluoromethyl)benzonitrile
4-[(cyclopropylmethyl)(propyl)amino]-2-nitrobenzonitrile
4-(diallylamino)-2-(trifluoromethyl)benzonitrile
5-[allyl(cyclopentyl)amino]-2-nitrobenzonitrile
4-[butyi(propyl)amino]-2-nitrobenzonitrile
4-[ethyl(2-methyl-2-propenyl)amino]-2-nitrobenzonitrile
4-[butyl(ethyl)amino]-2-(trifluoromethyl)benzonitrile
4-(dipropylamino)-2-(trifluoromethyl)benzonitrile
N-butyl-N-ethyl-3-methyl4-nitroaniline
5-(diallylamino)-2-nitrobenzonitrile
5-[(cyclopropylmethyl)(propyl)amino]-2-nitrobenzonitrile
4-(diallylamino)-2-nitrobenzonitrile
3-methyl-4-nitro-N,N-dipropylaniline
4-[sec-butyl(propyl)amino]-2-nitrobenzonitrile
5-[butyl(ethyl)amino]-2-nitrobenzonitrile
2-chloro-4-[(cyclopropylmethyl)(propyl)amino]benzonitrile
5-[butyl(propyl)amino]-2-nitrobenzonitrile
5-[(2-methoxyethyl)(methyl)amino]-2-nitrobenzonitrile
2-chloro4(diallylamino)benzonitrile
4-[(2-methoxyethyl)(propyl)amino]-2-nitrobenzonitrile
4-[allyl(cyclopentyl)amino]-2-nitrobenzonitrile
4-[ethyl(2-methyl-2-propenyl)amino]-2-(trifluoromethyl)benzonitrile
2-chloro-4-[[2-(dimethylamino)ethyl](methyl)amino]benzonitrile
N-(2-methoxyethyl)-N,2-dimethyl-4-nitroaniline
N-allyl-N-cyclopentyl-4-nitro-3-(trifluoromethyl)aniline
5-(dipropylamino)-2-nitrophenol
2-chloro-4-(dipropylamino)benzonitriie
5-[ethyl(2-methyl-2-propenyl)amino]-2-nitrobenzonitrile
N,N-diallyl-4-nitro-3-(trifluoromethyl)aniline
N,N-diallyl-2-methyl-4-nitroaniline
5-[(cyclopropylmethyl)(propyl)amino]-2-nitrophenol
N-(2-methoxyethyl)-3-methyl-4-nitro-N-propylaniline
4-[butyl(propyl)amino]-2-chlorobenzonitrile
N-butyl-2-chloro-N-methyl-4-nitroaniline
4-(dipropylamino)-2-nitrobenzonitrile
2-chloro-4-[(2-methoxyethyl)(propyl)amino]benzonitrile
N,N-diallyi-3-methyl-4-nitroaniline
N-(sec-butyl)-4-nitro-N-propyl-3-(trifluoromethyl)aniline
4-(dipentylamino)-2-nitrobenzonitrile
N-(cyclopropylmethyl)-3-methyl-4-nitro-N-propylaniline
5-(dipropylamino)-2-nitrobenzonitrile
N,N-dibutyl-4-nitro-3-(trifluoromethyl)aniline
N1-(2-chloro-4-nitrophenyl)-N1,N2,N2-trimethyl-1,2-ethanediamine
5-[sec-butyl(propyl)amino]-2-nitrobenzonitrile
N-ethyl-N-(2-methyl-2-propenyl)-4-nitro-3-(trifluoromethyl)aniline
N-(2-methoxyethyl)-4-nitro-N-propyl-3-(trifluoromethyl)aniline
4-[butyl(ethyl)amino]-2-chlorobenzonitrile
5-(dibutylamino)-2-nitrobenzonitrile
4-[butyl(ethyl)amino]-2-nitrobenzonitrile
4-[(2-methoxyethyl)(propyl)amino]-2-(trifluoromethyl)benzonitrile
5-[(2-methoxyethyl)(propyl)amino]-2-nitrobenzonitrile
4-[allyl(cyclopentyl)amino]-2-(trifluoromethyl)benzonitrile
4-[butyl(propyl)amino]-2-(trifluoromethyl)benzonitrile
N,N-dibutyl-3-methyl-4-nitroaniline
4-[methyl(octyl)amino]-2-nitrobenzonitrile
4-(dibutylamino)-2-(trifluoromethyl)benzonitrile
N-(cyclopropylmethyl)-4-nitro-N-propyl-2-(trifluoromethyl)aniline
N-allyl-N-cyclohexyl-4-nitro-3-(trifluoromethyl)aniline
4-[(2-methoxyethyl)(methyl)amino]-3-(trifluoromethyl)benzonitrile
4-(diallylamino)-3-nitrobenzonitrile
N-1-(2-chloro-4-nitrophenyl)-N1,N3,N3-trimethyl-1,3-propanediamine
N,N-bis(2-methoxyethyl)-4-nitro-3-(trifluoromethyl)aniline
4-[sec-butyl(propyl)amino]-2-(trifluoromethyl)benzonitrile
2-chloro-4-[ethyl(2-methyl-2-propenyl)amino]benzonitrile
N-cyclohexyl-N-ethyl-4-nitro-3-(trifluoromethyl)aniline
2-chloro-4-(dibutylamino)benzonitrile
4-[cyclohexyl(ethyl)amino]-2-nitrobenzonitrile
4-[bis(2-ethoxyethyl)amino]-3-chlorobenzonitrile
2-chloro-N-(2-methoxyethyl)-N-methyl-4-nitroaniline
N-butyl-N-ethyl-4-nitro-3-(trifluoromethyl)aniline
N-(sec-butyl)-3-methyl-4-nitro-N-propylaniline
N-(2-methoxyethyl)-N-methyl-4-nitro-2-(trifluoromethyl)aniline
4-{bis[3-(dimethylamino)propyl]amino}-2-(trifluoromethyl)benzonitrile
N-(cyclopropylmethyl)-4-nitro-N-propyl-3-(trifluoromethyl)aniline
4-[(methyl)(octyl)amino]-2-(trifluoromethyl)benzonitrile
4-(propylamino)-2-(trifluoromethyl)benzonitrile
4-nitro-N-propyl-3-(trifluoromethyl)aniline
3-{[4-nitro-3-(trifluoromethyl)phenyl]amino}propan-1-ol
4-[(3-hydroxypropyl)amino]-2-(trifluoromethyl)benzonitrile
4-{[2-hydroxy-1-(hydroxymethyl)ethyl]amino}-2-(trifluoromethyl)benzonitrile
4-(dimethylamino)-2-(trifluoromethyl)benzonitrile
4-(diethylamino)-2-(trifluoromethyl)benzonitrile
4-[methyl(2-methylpropyl)amino]-2-(trifluoromethyl)benzonitrile
4-[(cyclopropylmethyl)aminol]-2-(trifluoromethyl)benzonitrile
4-[(cyclopropylmethyl)(3-hydroxypropyl)amino]-2-(trifluoromethyl)benzonitrile
4-[(cyclopropylmethyl)(2-hydroxyethyl)amino]-2-(trifluoromethyl)benzonitrile
N-(cyclopropylmethyl)-4-nitro-3-(trifluoromethyl)aniline
3-{(cyclopropylmethyl)[4-nitro-3-(trifluoromethyl)phenyl]amino}-1-propanol
2-{(cyclopropylmethyl)[4-nitro-3-(trifluoromethyl)phenyl]amino}ethanol
4-[(cyclopropyl methyl)amino]-3-(trifluoromethyl)benzonitrile
1-[4-[(cyclopropylmethyl)(propyl)amino]-2-(trifluoromethyl)phenyl]ethanone
4-[(1-cyclopropylethyl)amino]-2-(trifluoromethyl)benzonitrile
4-[allyI(1-cyclopropylethyl)amino]-2-(trifluoromethyl)benzonitrile
4-[(2,2-dimethylpropyl)amino]-2-(trifluoromethyl)benzonitrile
4-[(2,2-dimethylpropyl)(3-hydroxypropyl)amino]-2-(trifluoromethyl)benzonitrile
4-[(2,2-dimethylpropyl)(2-propen-1 -yl)amino]-2-(trifluoromethyl)benzonitrile
4-[(2,3-dihydroxypropyl)(2,2-dimethylpropyl)amino]-2-(trifluoromethyl)benzonitrile
4-[(2,2-dimethylpropyl)(2-oxoethyl)amino]-2-(trifluoromethyl)benzonitrile
4-[(2,2-dimethylpropyl)(2-hydroxyethyl)amino]-2-(trifluoromethyl)benzonitrile
4-[(2,2-dimethylpropyl)(propyl)amino]-2-(trifluoromethyl)benzonitrile
4-[(1,1-dimethylethyl)amino]-2-(trifluoromethyl)benzonitrile
N-(1,1-dimethylethyl)-4-nitro-3-(trifluoromethyl)aniline
N-(cyclopropylmethyl)-N-(1,1-dimethylethyl)-4-nitro-3-(trifluoromethyl)aniline
4-[(1,1-dimethylethyl)(2-propen-1-yl)amino]-2-(trifluoromethyl)benzonitrile
4-[(1,1-dimethylethyl)(2-oxoethyl)amino]-2-(trifluoromethyl)benzonitrile
4-[(1,1-dimethylethyl)(propyl)amino]-2-(trifluoromethyl)benzonitrile
4-{(3-hydroxypropyl)[(1S)-1-methylpropyl]amino}-2-(trifluoromethyl)benzonitrile
2-(trifluoromethyl)-4-[(3,3,3-trifluoropropyl)amino]benzonitrile
4-[bis(2-fluoroethyl)amino]-2-(trifluoromethyl)benzonitrile
4-[(2,2,2-trifluoroethyl)amino]-2-(trifluoromethyl)benzonitrile
4-nitro-N-(2,2,2-trifluoroethyl)-3-(trifluoromethyl)aniline
4-[(3-hydroxypropyl)(2,2,2-trifluoroethyl)amino]-2-(trifluoromethyl)benzonitrile
4-[(3-hydroxypropyl)(2,2,2-trifluoroethyl)amino]-3-(trifluoromethyl)benzonitrile
4-bromo-N,N-bis(2,2,2-trifluoroethyl)-3-(trifluoromethyl)aniline
4-[bis(2,2,2-trifluoroethyl)amino]-2-(trifluoromethyl)benzonitrile
4-[(2,2-difluoroethyl)(2,2,2-trifluoroethyl)amino]-2-(trifluoromethyl)benzonitrile
bis(2,2,2-trifluoroethyl)[2-(trifluoromethyl)-4-biphenylyl]amine
4-[(2-hydroxyethyl)(2,2,2-trifluoroethyl)amino]-2-(trifluoromethyl)benzonitrile
4-[[2-(methyloxy)ethyl](2,2,2-trifluoroethyl)amino]-2-(trifluoromethyl)benzonitrile
4-[[2-(ethyloxy)ethyl](2,2,2-trifluoroethyl)amino]-2-(trifluoromethyl)benzonitrile
4-((2,2,2-trifluoroethyl){2-[(2,2,2-trifluoroethyl)oxy]ethyl}amino)-2-(trifluoromethyl)benzonitrile
4-[methyl(2,2,2-trifluoroethyl)amino]-2-(trifluoromethyl)benzonitrile
4-[ethyl(2,2,2-trifluoroethyl)amino]-2-(trifluoromethyl)benzonitrile
4-[propyl(2,2,2-trifluoroethyl)amino]-2-(trifluoromethyl)benzonitrile
4-[butyl(2,2,2-trifluoroethyl)amino]-2-(trifluoromethyl)benzonitrile
4-[(2-methylprop-2-enyl)(2,2,2-trifluoroethyl)amino]-2-(trifluoromethyl)benzonitrile
4-[isobutyl(2,2,2-trifluoroethyl)amino]-2-(trifluoromethyl)benzonitdle
4-[(3-methylbut-2-enyl)(2,2,2-trifluoroethyl)amino]-2-(trifluoromethyl)benzonitrile
4-[isopentyl(2,2,2-trifluoroethyl)amino]-2-(trifluoromethyl)benzonitrile
4-[prop-2-ynyl(2,2,2-trifluoroethyl)amino]-2-(trifluoromethyl)benzonitrile
4-[(2-fluoroethyl)(2,2,2-trifluoroethyl)amino]-2-(trifluoromethyl)benzonitrile
4-[[2-(methylthio)ethyl](2,2,2-trifluoroethyl)amino]-2-(trifluoromethyl)benzonitrlle
4-[(2-azidoethyl)(2,2,2-trifluoroethyl)amino]-2-(trifluoromethyl)benzonitrile.
N-{2-[[4-cyano-3-(trifluoromethyl)phenyl](2,2,2-trifluoroethyl)amino]ethyl}acetamide methyl 2-[[4-cyano-3-(trifluoromethyl)phenyl](2,2,2-trifluoroethyl)amino]ethylcarbamate
tert-butyl 2-[[4-cyano-3-(trifluoromethyl)phenyl](2,2,2-trifluoroethyl)amino]ethylcarbamate
N-{2-[[4-cyano-3-(trifluoromethyl)phenyl](2,2,2-trifluoroethyl)amino]ethyl}methanesulfonamide; and
4-(dipropylamino)phthalonitrile.

Another aspect of the present invention includes compounds of formula (I): 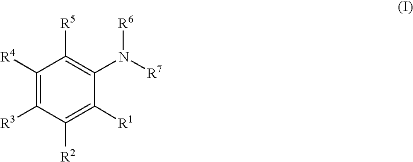
including salts, solvates, and physiologically functional derivatives thereof, wherein
R¹ is H, cyano, nitro, halogen, haloalkyl, alkyl, alkenyl, alkynyl, hydroxy, alkoxy, haloalkoxy, —OC(O)R⁸, or aryl;
R² is H, cyano, nitro, halogen, haloalkyl, alkyl, alkenyl, alkynyl, hydroxy, alkoxy, haloalkoxy, —OC(O)R⁸, or aryl;
R³is cyano, nitro, halogen, haloalkyl, heterocyclyl, hydroxy, alkoxy, haloalkoxy, —OC(O)R⁸, —C(O)₂R⁸, —CONHR⁸, —C(O)R⁸, —S(O)ₙR⁸, —SO₂N(R⁸)₂, —NHC(O)R⁸, or —NHSO₂R⁸;
R⁴ is H, cyano, nitro, halogen, haloalkyl, alkyl, alkenyl, alkynyl, hydroxy, alkoxy, haloalkoxy, —OC(O)R⁸, or aryl;
R⁵ is H, cyano, nitro, halogen, haloalkyl, alkyl, alkenyl, alkynyl, hydroxy, alkoxy, haloalkoxy, —OC(O)R⁸, or aryl;
each of R⁸ and R⁷ independently are selected from H or —(R^{a})ₓ-R⁹;
R^{a} is a C₁-C₈ alkylene chain where x is 0 or 1;
each R⁸ independently is H, alkyl, alkenyl, alkynyl, cycloalkyl, heterocyclyl, aryl, aralkyl, heteroaryl, or heteroaralkyl;
R⁹ is alkyl, haloalkyl, hydroxyalkyl, alkenyl, alkynyl, alkoxy, alkylthio, haloalkoxy, cycloalkyl, formyl, azido, or —NR¹⁰R¹¹;
R¹⁰ and R¹¹ each independently are H, alkyl, —C(O)H, —C(O)R¹², —C(O)OR¹², or —SO₂R¹²; and
R¹² is alkyl.

Thus, in this aspect of the invention, each of R¹, R², R⁴, and R⁵ may each be H. Preferred compounds include:
N,N-diallyl-4-nitroaniline
N-(cyclopropylmethyl)-4-nitro-N-propylaniline
4-(dipropylamino)benzonitrile; or
4-nitro-N,N-dipropylaniline.

Another aspect of the present invention includes a compound substantially as hereinbefore defined with reference to any one of the Examples.

Another aspect of the present invention includes a pharmaceutical composition comprising a compound of the present invention and a pharmaceutically acceptable carrier.

Another aspect of the present invention includes a compound of the present invention for use as an active therapeutic substance.

Another aspect of the present invention includes a compound of the present invention for use in the treatment or prophylaxis of conditions or disorders that respond to selective androgen receptor modulation.

Another aspect of the present invention includes a compound of the present invention for use in the treatment or prophylaxis of osteoporosis, muscle wasting, frailty, cardiovascular disease, breast cancer, uterine cancer, prostate hyperplasia, prostate cancer, dyslipidemia, menopausal vasomotor conditions, urinary incontinence, artherosclerosis, libido enhancement, depression, uterine fibroid disease, aortic smooth muscle cell proliferation, endometriosis, or ADAM.

Another aspect of the present invention includes the use of a compound of the present invention in the manufacture of a medicament for use in the treatment or prophylaxis of conditions or disorders that respond to selective androgen receptor modulation.

Another aspect of the present invention includes using a compound according to the present invention in the manufacture of a medicament for use in the treatment or prophylaxis of osteoporosis, muscle wasting, frailty, cardiovascular disease, breast cancer, uterine cancer, prostatic hyperplasia, prostate cancer, dyslipidemia, menopausal vasomotor conditions, urinary incontinence, artherosclerosis, libido enhancement, depression, uterine fibroid disease, aortic smooth muscle cell proliferation, endometriosis, or ADAM.

Another aspect of the present invention includes a method for the treatment or prophylaxis of conditions or disorders that respond to selective androgen receptor modulation comprising the administration of a compound according to the present invention.

Another aspect of the present invention includes a method for the treatment or prophylaxis of osteoporosis, muscle wasting, frailty, cardiovascular disease, breast cancer, uterine cancer, prostatic hyperplasia, prostate cancer, dyslipidemia, menopausal vasomotor conditions, urinary incontinence, artherosclerosis, libido enhancement, depression, uterine fibroid disease, aortic smooth muscle cell proliferation, endometriosis, or ADAM comprising the administration of a compound according to the present invention.

The compounds of the present invention modulate the function of the nuclear hormone receptors, particularly the androgen receptor (“AR”). The present invention includes compounds that are selective agonists, partial agonists, antagonists, or partial antagonists of the AR. Compounds of the present invention are useful in the treatment of AR-associated diseases and conditions, for example, a disease or condition that is prevented, alleviated, or cured through the modulation of the function or activity of AR. Such modulation may be isolated within certain tissues or widespread throughout the body of the subject being treated.

An aspect of the present invention is the use of the compounds of the present invention for the treatment or prophylaxis of a variety of disorders including, but not limited to, osteoporosis and/or the prevention of reduced bone mass, density, or growth, osteoarthritis, acceleration of bone fracture repair and healing, acceleration of healing in joint replacement, periodontal disease, acceleration of tooth repair or growth, Paget's disease, osteochondrodysplasias, muscle wasting, the maintenance and enhancement of muscle strength and function, frailty or age-related functional. decline (“ARFD”), dry eye, sarcopenia, chronic fatigue syndrome, chronic myaligia, acute fatigue syndrome, acceleration of wound healing, maintenance of sensory function, chronic liver disease, AIDS, weightlessness, burn and trauma recovery, thrombocytopenia, short bowel syndrome, irritable bowel syndrome, inflammatory bowel disease, Crohn's disease and ulcerative colitis, obesity, eating disorders including anorexia associated with cachexia or aging, hypercortisolism and Cushing's syndrome, cardiovascular disease or cardiac dysfunction, congestive heart failure, high blood pressure, malignant tumor cells containing the androgen receptor including breast, brain, skin, ovary, bladder, lymphatic, liver, kidney, uterine, pancreas, endometrium, lung, colon, and prostate, prostatic hyperplasia, hirsutism, acne, seborrhea, androgenic alopecia, anemia, hyperpilosity, adenomas and neoplasis of the prostate, hyperinsulinemia, insulin resistance, diabetes, syndrome X, dyslipidemia, menopausal vasomotor conditions, urinary incontinence, artherosclerosis, libido enhancement, sexual dysfunction, depression, nervousness, irritability, stress, reduced mental energy and low self-esteem, improvement of cognitive function, endometriosis, polycystic ovary syndrome, counteracting preeclampsia, premenstral syndrome, contraception, uterine fibroid disease, aortic smooth muscle cell proliferation, male hormone replacement, or ADAM.

### DETAILED DESCRIPTION OF THE PREFERRED EMBODIMENT

Terms are used within their accepted meanings. The following definitions are meant to clarify, but not limit, the terms defined.

As used herein the term “alkyl” refers to a straight or branched chain hydrocarbon, preferably having from one to twelve carbon atoms, which may be optionally substituted, with multiple degrees of substitution included within the present invention. Examples of “alkyl” as used herein include, but are not limited to, methyl, ethyl, propyl, isopropyl, isobutyl, n-butyl, tert-butyl, isopentyl, n-pentyl, and substituted versions thereof.

As used throughout this specification, the preferred number of atoms, such as carbon atoms, will be represented by, for example, the phrase “Cₓ₋C_{y} alkyl,” which refers to an alkyl group, as herein defined, containing the specified number of carbon atoms. Similar terminology will apply for other preferred terms and ranges as well.

As used herein the term “alkenyl” refers to a straight or branched chain aliphatic hydrocarbon containing one or more carbon-to-carbon double bonds that may be optionally substituted, with multiple degrees of substitution included within the present invention. Examples include, but are not limited to, vinyl and the like and substituted versions thereof.

As used herein the term “alkynyl” refers to a straight or branched chain aliphatic hydrocarbon containing one or more carbon-to-carbon triple bonds that may be optionally substituted, with multiple degrees of substitution included within the present invention. Examples include, but are not limited to, ethynyl and the like and substituted versions thereof.

As used herein, the term “alkylene” refers to a straight or branched chain divalent hydrocarbon radical, preferably having from one to ten carbon atoms. Alkylene groups as defined herein may optionally be substituted, with multiple degrees of substitution included within the present invention. Examples of “alkylene” as used herein include, but are not limited to, methylene (—CH₂-), ethylene (—CH₂-CH₂-), and branched/substituted versions thereof.

As used herein, the term “cycloalkyl” refers to an optionally substituted non-aromatic cyclic hydrocarbon ring, which optionally includes an alkylene linker through which the cycloalkyl may be attached, with multiple degrees of substitution included within the present invention. Exemplary “cycloalkyl” groups include, but are not limited to, cyclopropyl, cyclobutyl, cyclopentyl, cyclohexyl, cycloheptyl, and substituted versions thereof.

As used herein, the terms “heterocycle,” “heterocyclic,” or “heterocyclyl” refers to a mono- or poly-cyclic ring system containing optionally one or more degrees of unsaturation, but not to overlap with heteroaryl, and also containing optionally one or more heteroatoms. Preferred heteroatoms include N, O, and/or S, including N-oxides, sulfur oxides, and dioxides. Preferably the ring is three to ten-membered and is either saturated or has one or more degrees of unsaturation. Optionally, as used herein, the heterocycle may be substituted, with multiple degrees of substitution being allowed. Such rings may be optionally fused to one or more of another “heterocyclic” ring(s), heteroaryl ring(s), aryl ring(s), or cycloalkyl ring(s). Examples of “heterocyclic” groups include, but are not limited to, tetrahydrofuran, pyran, 1,4-dioxane, 1,3-dioxane, piperidine, pyrrolidine, morpholine, tetrahydrothiopyran, and tetrahydrothiophene.

As used herein, the term “aryl” refers to an optionally substituted benzene ring or to an optionally substituted fused benzene ring system, for example anthracene, phenanthrene, or naphthalene ring systems. Multiple degrees of substitution are included within the present definition. Examples of “aryl” groups include, but are not limited to, phenyl, 2-naphthyl, 1-naphthyl, biphenyl, and substituted derivatives thereof. Similarly, the term “aralkyl” refers to an aryl group attached through an alkylene linker, such as benzyl and the like.

As used herein, the term “heteroaryl” refers to an optionally substituted monocyclic five to seven membered aromatic ring, or to an optionally substituted fused bicyclic aromatic ring system comprising two of such aromatic rings, which contain one or more nitrogen, sulfur, and/or oxygen atoms, where N-oxides, sulfur oxides, and dioxides are permissible heteroatom substitutions. Multiple degrees of substitution are included within the present definition. Examples of “heteroaryl” groups used herein include, but should not be limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, and substituted versions thereof. Similarly, the term “heteroaralkyl” refers to a heteroayl group attached through an alkylene linker.

As used herein the term “halogen” refers to fluorine, chlorine, bromine, or iodine.

As used herein the term “haloalkyl” refers to an alkyl group, as defined herein that is substituted with at least one halogen. Examples of branched or straight chained “haloalkyl” groups useful in the present invention include, but are not limited to, methyl, ethyl, propyl, isopropyl, n-butyl, and t-butyl substituted independently with one or more halogens, e.g., fluoro, chloro, bromo, and iodo. The term “haloalkyl” should be interpreted to include such substituents such as —CF₃, —CH₂—CH₂—F, and the like.

As used herein the term “hydroxy” refers to the group —OH.

As used herein the term “formyl” refers to the group 13 C(O)H.

As used herein the term “hydroxyalkyl” refers to a group —Rₐ—OH, where Rₐ is an alkylene as defined above.

As used herein the term “alkoxy” refers to a group —ORₐ, where Rₐ is alkyl as defined above.

As used herein the term “alkylthio” refers to a group —SRₐ, where Rₐ is alkyl as defined above.

As used herein the term “aryloxy” refers to a group —OR_{b}, where R_{b} is aryl as defined above.

As used herein the term “haloalkoxy” refers to a group —ORₐ, where Rₐ is haloalkyl as defined above.

As used herein the term “nitro” refers to the group —NO₂.

As used herein the term “cyano” refers to the group —CN.

As used herein the term “azido” refers to the group —N₃.

As used herein the term “amino” refers to the group —NH₂, and “substituted amino” refers to a group —N(Rₐ)(R_{b}), where one of Rₐ and R_{b} are other than H. For example, “substituted amino” includes the groups —N(CH₃)(CH₃), —N(CH₃)(CH₂—CH₃), and the like.

As used herein throughout the present specification, the phrase “optionally substituted” or variations thereof denote an optional substitution, including multiple degrees of substitution, with one or more substituent group. The phrase should not be interpreted so as to be imprecise or duplicative of substitution patterns herein described or depicted. Rather, those of ordinary skill in the art will appreciate that the phrase is included to provide for obvious modifications, which are encompassed within the scope of the appended claims.

Exemplary optional substituent groups include acyl; alkyl; alkenyl; alkynyl; alkylsulfonyl; alkoxy; cyano; halogen; haloalkyl; hydroxy; nitro; aryl, which may be further substituted with acyl, alkoxy, alkyl, alkenyl, alkynyl, alkylsulfonyl, cyano, halogen, haloalkyl, hydroxy, or nitro; heteroaryl, which may be further substituted with acyl, alkoxy, alkyl, alkenyl, alkynyl, alkylsulfonyl, cyano, halogen, haloalkyl, hydroxy, or nitro; arylsulfonyl, which may be further substituted with acyl, alkoxy, alkyl, alkenyl, alkynyl, alkylsulfonyl, cyano, halogen, haloalkyl, hydroxy, or nitro; heteroarylsulfonyl, which may be further substituted with acyl, alkoxy, alkyl, alkenyl, alkynyl, alkylsulfonyl, cyano, halogen, haloalkyl, hydroxy, or nitro; aryloxy, which may be further substituted with acyl, alkoxy, alkyl, alkenyl, alkynyl, alkylsulfonyl, cyano, halogen, haloalkyl, hydroxy, or nitro; heteroaryioxy, which may be further substituted with acyl, alkoxy, alkyl, alkenyl, alkynyl, alkylsulfonyl, cyano, halogen, haloalkyl, hydroxy, or nitro; or —N(R^{★})₂; where for each occurrence R★ is independently selected from H, alkyl, alkenyl, alkynyl, cycloalkyl, heterocyclyl, aryl, aralkyl, heteroaryl, heteroaralkyl, alkylsulfonyl, arylsulfonyl, or heteroarylsulfonyl, where each occurrence of such aryl or heteroaryl may be substituted with one or more acyl, alkoxy, alkyl, alkenyl, alkylsulfonyl, cyano, halogen, haloalkyl, hydroxy, or nitro, or the two R★ may combine to form a ring, optionally having additional heteroatoms, optionally having one or more degrees of unsaturation, and optionally being further substituted with acyl, alkoxy, alkyl, alkenyl, alkynyl, alkylsulfonyl, cyano, halogen, haloalkyl, hydroxy, or nitro.

The compounds of formulas (I) may crystallize in more than one form, a characteristic known as polymorphism, and such polymorphic forms (“polymorphs”) are within the scope of formula (I). Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically/diastereomerically enriched mixtures. Also included within the scope of the invention are the individual isomers of the compounds represented by formula (I), as well as any wholly or partially equilibrated mixtures thereof. The present invention also includes the individual isomers of the compounds represented by the formulas above as mixtures with isomers thereof in which one or more chiral centers are inverted.

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. Representative salts include acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. Other salts, which are not pharmaceutically acceptable, may be useful in the preparation of compounds of this invention and these should be considered to form a further aspect of the invention.

As used herein, the term “solvate” refers to a complex of variable stoichiometry formed by a solute (in this invention, a compound of Formula I, or a salt or physiologically functional derivative thereof) and a solvent. Such solvents, for the purpose of the invention, should not interfere with the biological activity of the solute. Non-limiting examples of suitable solvents include, but are not limited to water, methanol, ethanol, and acetic acid. Preferably the solvent used is a pharmaceutically acceptable solvent. Non-limiting examples of suitable pharmaceutically acceptable solvents include water, ethanol, and acetic acid. Most preferably the solvent used is water.

As used herein, the term “physiologically functional derivative” refers to any pharmaceutically acceptable derivative of a compound of the present invention that, upon administration to a mammal, is capable of providing (directly or indirectly) a compound, of the present invention or an active metabolite thereof. Such derivatives, for example, esters and amides, will be clear to those skilled in the art, without undue experimentation. Reference may be made to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “effective amount” means that amount of a drug or pharmaceutical agent that will elicit the biological or medical response of a tissue, system, animal, or human that is being sought, for instance, by a researcher or clinician. The biological or medical response may be considered a prophylactic response or a treatment response. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a compound of formula (I), as well as salts, solvates, and physiological functional derivatives thereof, may be administered as the raw chemical. Additionally, the active ingredient may be presented as a pharmaceutical composition.

Accordingly, the invention further provides pharmaceutical compositions that include effective amounts of compounds of the formula (I) and salts, solvates, and physiological functional derivatives thereof, and one or more pharmaceutically acceptable carriers, diluents, or excipients. The compounds of formula (I) and salts, solvates, and physiologically functional derivatives thereof, are as herein described. The carrier(s), diluent(s) or excipient(s) must be acceptable, in the sense of being compatible with the other ingredients of the formulation and not deleterious to the recipient of the pharmaceutical composition.

In accordance with another aspect of the invention there is also provided a process for the preparation of a pharmaceutical formulation including admixing a compound of the formula (I) or salts, solvates, and physiological functional derivatives thereof, with one or more pharmaceutically acceptable carriers, diluents or excipients.

A therapeutically effective amount of a compound of the present invention will depend upon a number of factors. For example, the species, age, and weight of the recipient, the precise condition requiring treatment and its severity, the nature of the formulation, and the route of administration are all factors to be considered. The therapeutically effective amount ultimately should be at the discretion of the attendant physician or veterinarian. Regardless, an effective amount of a compound of formula (I) for the treatment of humans suffering from frailty, generally, should be in the range of 0.1 to 100 mg/kg body weight of recipient (mammal) per day. More usually the effective amount should be in the range of 1 to 10 mg/kg body weight per day. Thus, for a 70 kg adult mammal the actual amount per day would usually be from 70 to 700 mg. This amount may be given in a single dose per day or in a number (such as two, three, four, five, or more) of sub-doses per day such that the total daily dose is the same. An effective amount of a salt, solvate, or physiologically functional derivative thereof, may be determined as a proportion of the effective amount of the compound of formula (I) per se. Similar dosages should be appropriate for treatment or prophylaxis of the other conditions referred to herein.

Pharmaceutical formulations may be presented in unit dose forms containing a predetermined amount of active ingredient per unit dose. Such a unit may contain, as a non-limiting example, 0.5 mg to 1 g of a compound of the formula (I), depending on the condition being treated, the route of administration, and the age, weight, and condition of the patient. Preferred unit dosage formulations are those containing a daily dose or sub-dose, as herein above recited, or an appropriate fraction thereof, of an active ingredient. Such pharmaceutical formulations may be prepared by any of the methods well known in the pharmacy art.

Pharmaceutical formulations may be adapted for administration by any appropriate route, for example by an oral (including buccal or sublingual), rectal, nasal, topical (including buccal, sublingual or transdermal), vaginal, or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions, each with aqueous or non-aqueous liquids; edible foams or whips; or oil-in-water liquid emulsions or water-in-oil liquid emulsions. For instance, for oral administration in the form of a tablet or capsule, the active drug component can be combined with an oral, non-toxic pharmaceutically acceptable inert carrier such as ethanol, glycerol, water, and the like. Generally, powders are prepared by comminuting the compound to a suitable fine size and mixing with an appropriate pharmaceutical carrier such as an edible carbohydrate, as, for example, starch or mannitol. Flavorings, preservatives, dispersing agents, and coloring agents can also be present.

Capsules are made by preparing a powder, liquid, or suspension mixture and encapsulating with gelatin or some other appropriate shell material. Glidants and lubricants such as colloidal silica, talc, magnesium stearate, calcium stearate, or solid polyethylene glycol can be added to the mixture before the encapsulation. A disintegrating or solubilizing agent such as agar-agar, calcium carbonate or sodium carbonate can also be added to improve the availability of the medicament when the capsule is ingested. Moreover, when desired or necessary, suitable binders, lubricants, disintegrating agents, and coloring agents can also be incorporated into the mixture. Examples of suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants useful in these dosage forms include, for example, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Tablets are formulated, for example, by preparing a powder mixture, granulating or slugging, adding a lubricant and disintegrant, and pressing into tablets. A powder mixture may be prepared by mixing the compound, suitably comminuted, with a diluent or base as described above. Optional ingredients include binders such as carboxymethylcellulose, aliginates, gelatins, or polyvinyl pyrrolidone, solution retardants such as paraffin, resorption accelerators such as a quaternary salt, and/or absorption agents such as bentonite, kaolin, or dicalcium phosphate. The powder mixture can be wet-granulated with a binder such as syrup, starch paste, acadia mucilage or solutions of cellulosic or polymeric materials, and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solutions, syrups, and elixirs can be prepared in dosage unit form so that a given quantity contains a predetermined amount of the compound. Syrups can be prepared, for example, by dissolving the compound in a suitably flavored aqueous solution, while elixirs are prepared through the use of a non-toxic alcoholic vehicle. Suspensions can be formulated generally by dispersing the compound in a non-toxic vehicle. Solubilizers and emulsifiers such as ethoxylated isostearyl alcohols and polyoxy ethylene sorbitol ethers, preservatives; flavor additives such as peppermint oil, or natural sweeteners, saccharin, or other artificial sweeteners; and the like can also be added.

Where appropriate, dosage unit formulations for oral administration can be microencapsulated. The formulation can also be prepared to prolong or sustain the release as for example by coating or embedding particulate material in polymers, wax or the like.

The compounds of formula (I) and salts, solvates, and physiological functional derivatives thereof, can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The compounds of formula (I) and salts, solvates, and physiologically functional derivatives thereof may also be delivered by the use of monoclonal antibodies as individual carriers to which the compound molecules are coupled.

The compounds may also be coupled with soluble polymers as targetable drug carriers. Such polymers can include polyvinylpyrrolidone (PVP), pyran copolymer, polyhydroxypropylmethacrylamide-phenol, polyhydroxyethyl-aspartamidephenol, or polyethyleneoxidepolylysine substituted with palmitoyl residues. Furthermore, the compounds may be coupled to a class of biodegradable polymers useful in achieving controlled release of a drug; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by lontophoresis as generally described in Pharmaceutical Research, 3(6), 318 (1986), incorporated herein by reference as related to such delivery systems.

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles, and mouthwashes.

Pharmaceutical formulations adapted for nasal administration, where the carrier is a solid, include a coarse powder having a particle size for example in the range 20 to 500 microns. The powder is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered dose pressurized aerosols, nebulizers, or insufflators.

Pharmaceutical formulations adapted for rectal administration may be presented as suppositories or as enemas.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

In addition to the ingredients particularly mentioned above, the formulations may include other agents conventional in the art having regard to the type of formulation in question. For example, formulations suitable for oral administration may include flavoring or coloring agents.

The compounds of the present invention and their salts, solvates, and physiologically functional derivatives thereof, may be employed alone or in combination with other therapeutic agents for the treatment of the above-mentioned conditions. For example, in frailty therapy, combination may be had with other anabolic or osteoporosis therapeutic agents. As one example, osteoporosis combination therapies according to the present invention would thus comprise the administration of at least one compound of formula (I) or a salt, solvate, or physiologically functional derivative thereof, and the use of at least one other osteoporosis therapy. As a further example, combination therapies according to the present invention include the administration of at least one compound of formula (I) or a salt, solvate, or physiologically functional derivative thereof, and at least one other osteoporosis treatment agent, for example, an anti-bone resorption agent. The compound(s) of formula (I) and the other pharmaceutically active agent(s) may be administered together or separately and, when administered separately, administration may occur simultaneously or sequentially, in any order. The amounts of the compound(s) of formula (I) and the other pharmaceutically active agent(s) and the relative timings of administration will be selected in order to achieve the desired combined therapeutic effect. The administration in combination of a compound of formula (I) salts, solvates, or physiologically functional derivatives thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time.

Another potential osteoporosis treatment agent is a bone building (anabolic) agent. Bone building agents can lead to increases in parameters such as bone mineral density that are greater than those than can be achieved with anti-resorptive agents. In some cases, such anabolic agents can increase trabecular connectivity leading to greater structural integrity of the bone.

Other potential therapeutic combinations include the compounds of the present invention combined with other compounds of the present invention, growth promoting agents, growth hormone secretagogues, growth hormone releasing factor and its analogs, growth hormone and its analogs, somatomedins, alpha-ardenergic agonists, serotonin 5-HT_{D} agonists, agents that inhibit somatostatin or its release, 5-α-reductase inhibitors, aromatase inhibitors, GnRH agonists or antagonists, parathyroid hormone, bisphosphonates, estrogen, testosterone, SERMs, progesterone receptor agonists or antagonists, and/or with other modulators of nuclear hormone receptors.

One skilled in the art will acknowledge that although the compounds embodied herein will be used as selective agonists, partial agonists, and antagonists, compounds with mixed steroid activities may also be employed.

The compounds of the present invention may be used in the treatment of a variety of disorders and conditions and, as such, the compounds of the present invention may be used in combination with a variety of other suitable therapeutic agents useful in the treatment or prophylaxis of those disorders or conditions. Non-limiting examples include combinations of the present invention with anti-diabetic agents, anti-osteoporosis agents, anti-obesity agents, anti-inflammatory agents, anti-anxiety agents, anti-depressants, anti-hypertensive agents, anti-platelet agents, anti-thrombotic and thrombolytic agents, cardiac glycosides, cholesterol or lipid lowering agents, mineralocorticoid receptor antagonists, phosphodiesterase inhibitors, kinase inhibitors, thyroid mimetics, anabolic agents, viral therapies, cognitive disorder therapies, sleeping disorder therapies, sexual dysfunction therapies, contraceptives, cytotoxic agents, radiation therapy, anti-proliferative agents, and anti-tumor agents. Additionally, the compounds of the present invention may be combined with nutritional supplements such as amino acids, triglycerides, vitamins, minerals, creatine, piloic acid, carnitine, or coenzyme Q10.

An aspect of the present invention is the use of the compounds of the present invention for the treatment or prophylaxis of a variety of disorders including, but not limited to, osteoporosis and/or the prevention of reduced bone mass, density, or growth, osteoarthritis, acceleration of bone fracture repair and healing, acceleration of healing in joint replacement, periodontal disease, acceleration of tooth repair or growth, Paget's disease, osteochondrodysplasias, muscle wasting, the maintenance and enhancement of muscle strength and function, frailty or age-related functional decline (“ARFD”), dry eye, sarcopenia, chronic fatigue syndrome, chronic myaligia, acute fatigue syndrome, acceleration of wound healing, maintenance of sensory function, chronic liver disease, AIDS, weightlessness, burn and trauma recovery, thrombocytopenia, short bowel syndrome, irritable bowel syndrome, inflammatory bowel disease, Crohn's disease and ulcerative colitis, obesity, eating disorders including anorexia associated with cachexia or aging, hypercortisolism and Cushing's syndrome, cardiovascular disease or cardiac dysfunction, congestive heart failure, high blood pressure, malignant tumor cells containing the androgen receptor including breast, brain, skin, ovary, bladder, lymphatic, liver, kidney, uterine, pancreas, endometrium, lung, colon, and prostate, prostatic hyperplasia, hirsutism, acne, seborrhea, androgenic alopecia, anemia, hyperpilosity, adenomas and neoplasis of the prostate, hyperinsulinemia, insulin resistance, diabetes, syndrome X, dyslipidemia, menopausal vasomotor conditions, urinary incontinence, artherosclerosis, libido enhancement, sexual dysfunction, depression, nervousness, irritability, stress, reduced mental energy and low self-esteem, improvement of cognitive function, endometriosis, polycystic ovary syndrome, counteracting preeclampsia, premenstral syndrome, contraception, uterine fibroid disease, aortic smooth muscle cell proliferation, male hormone replacement, or ADAM.

In particular, the compounds of the present invention are believed useful, either alone or in combination with other agents, in the treatment of and use as male and female hormone replacement therapy, hypogonadism, osteoporosis, muscle wasting, wasting diseases, cancer cachexia, frailty, prostatic hyperplasia, prostate cancer, breast cancer, menopausal and andropausal vasomotor conditions, urinary incontinence, sexual dysfunction, erectile dysfunction, depression, uterine fibroid disease, and/or endometriosis, treatment of acne, hirsutism, stimulation of hematopoiesis, male contraception, impotence, and as anabolic agents.

Another aspect of the present invention thus also provides compounds of formula (I) and salts, solvates, or physiologically functional derivatives thereof, for use in medical therapy. Particularly, the present invention provides for the treatment or prophylaxis of disorders mediated by androgenic activity. More particularly, the present invention provides through the treatment or prophylaxis of disorders responsive to tissue-selective anabolic and or androgenic activity. A further aspect of the invention provides a method of treatment or prophylaxis of a mammal suffering from a disorder mediated by androgenic activity, which includes administering to said subject an effective amount of a compound of formula (I) or a salt, solvate, or physiologically functional derivative thereof.

A further aspect of the invention provides a method of treatment or prophylaxis of a mammal requiring the treatment or prophylaxis of a variety of disorders including, but not limited to, osteoporosis and/or the prevention of reduced bone mass, density, or growth, osteoarthritis, acceleration of bone fracture repair and healing, acceleration of healing in joint replacement, periodontal disease, acceleration of tooth repair or growth, Paget's disease, osteochondrodysplasias, muscle wasting, the maintenance and enhancement of muscle strength and function, frailty or age-related functional decline (“ARFD”), dry eye, sarcopenia, chronic fatigue syndrome, chronic myaligia, acute fatigue syndrome, acceleration of wound healing, maintenance of sensory function, chronic liver disease, AIDS, weightlessness, burn and trauma recovery, thrombocytopenia, short bowel syndrome, irritable bowel syndrome, inflammatory bowel disease, Crohn's disease and ulcerative colitis, obesity, eating disorders including anorexia associated with cachexia or aging, hypercortisolism and Cushing's syndrome, cardiovascular disease or cardiac dysfunction, congestive heart failure, high blood pressure, malignant tumor cells containing the androgen receptor including breast, brain, skin, ovary, bladder, lymphatic, liver, kidney, uterine, pancreas, endometrium, lung, colon, and prostate, prostatic hyperplasia, hirsutism, acne, seborrhea, androgenic alopecia, anemia, hyperpilosity, adenomas and neoplasis of the prostate, hyperinsulinemia, insulin resistance, diabetes, syndrome X, dyslipidemia, menopausal vasomotor conditions, urinary incontinence, artherosclerosis, libido enhancement, sexual dysfunction, depression, nervousness, irritability, stress, reduced mental energy and low self- esteem, improvement of cognitive function, endometriosis, polycystic ovary syndrome, counteracting preeclampsia, premenstral syndrome, contraception, uterine fibroid disease, aortic smooth muscle cell proliferation, male hormone replacement, or ADAM. Preferably the compounds of the present invention are used as male and female hormone replacement therapy or for the treatment or prevention of hypogonadism, osteoporosis, muscle wasting, wasting diseases, cancer cachexia, frailty, prostatic hyperplasia, prostate cancer, breast cancer, menopausal and andropausal vasomotor conditions, urinary incontinence, sexual dysfunction, erectile dysfunction, depression, uterine fibroid disease, and/or endometriosis, treatment of acne, hirsutism, stimulation of hematopoiesis, male contraception, impotence, and as anabolic agents, which use includes administering to a subject an effective amount of a compound of formula (I) or a salt, solvate, or physiologically functional derivative thereof. The mammal requiring treatment with a compound of the present invention is typically a human being.

The compounds of this invention may be made by a variety of methods, including well-known standard synthetic methods. Illustrative general synthetic methods are set out below and then specific compounds of the invention are prepared in the working Examples.

In all of the schemes described below, protecting groups for sensitive or reactive groups are employed where necessary in accordance with general principles of synthetic chemistry. Protecting groups are manipulated according to standard methods of organic synthesis (T. W. Green and P. G. M. Wuts (1991) Protecting Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of formula (I).

Those skilled in the art will recognize if a stereocenter exists in compounds of formula (I). Accordingly, the present invention includes all possible stereoisomers and includes not only racemic compounds but the individual enantiomers as well. When a compound is desired as a single enantiomer, such may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-lnterscience, 1994), incorporated by reference with regard to stereochemistry.

Representative AR modulator compounds, agonists, partial agonists, and antagonists according to the current invention include:
4-[(Cyclopropylmethyl)(propyl)amino]-2-(trifluoromethyl)benzonitrile
4-[(cyclopropylmethyl)(propyl)amino]-2-nitrobenzonitrile
4-(diallylamino)-2-(trifluoromethyl)benzonitrile
5-[allyl(cyclopentyl)amino]-2-nitrobenzonitrile
4-[butyl(propyl)amino]-2-nitrobenzonitrile
4-[ethyl(2-methyl-2-propenyl)amino]-2-nitrobenzonitrile
4-[butyl(ethyl)amino]-2-(trifluoromethyl)benzonitrile
4-(dipropylamino)-2-(trifluoromethyl)benzonitrile
N-butyl-N-ethyl-3-methyl-4-nitroaniline
5-(diallylamino)-2-nitrobenzonitrile
5-[(cyclopropylmethyl)(propyl)amino]-2-nitrobenzonitrile
4-(diallylamino)-2-nitrobenzonitrile
3-methyl-4-nitro-N,N-dipropylaniline
4-[sec-butyl(propyl)amino]-2-nitrobenzonitrile
5-[butyl(ethyl)amino]-2-nitrobenzonitrile
2-chloro-4-[(cyclopropylmethyl)(propyl)amino]benzonitrile
5-[butyl(propyl)amino]-2-nitrobenzonitrile
5-[(2-methoxyethyl)(methyl)amino]-2-nitrobenzonitrile
2-chloro-4-(diallylamino)benzonitrile
4-[(2-methoxyethyl)(propyl)amino]-2-nitrobenzonitrile
4-[allyl(cyclopentyl)amino]-2-nitrobenzonitrile
4-[ethyl(2-methyl-2-propenyl)amino]-2-(trifluoromethyl)benzonitrile
2-chloro-4-[[2-(dimethylamino)ethyl](methyl)amino]benzonitrile
N-(2-methoxyethyl)-N,2-dimethyl-4-nitroaniline
N-allyl-N-cyclopentyl-4-nitro-3-(trifluoromethyl)aniline
5-(dipropylamino)-2-nitrophenol
2-chloro-4-(dipropylamino)benzonitrile
5-[ethyl(2-methyl-2-propenyl)amino]-2-nitrobenzonitrile
N,N-diallyl-4-nitro-3-(trifluoromethyl)aniline
N,N-diallyl-2-methyl-4-nitroaniline
5-[(cyclopropylmethyl)(propyl)amino]-2-nitrophenol
N-(2-methoxyethyl)-3-methyl-4-nitro-N-propylaniline
4-[butyl(propyl)amino]-2-chlorobenzonitrile
N-butyl-2-chloro-N-methyl-4-nitroaniline
4-(dipropylamino)-2-nitrobenzonitrile
2-chloro-4-[(2-methoxyethyl)(propyl)amino]benzonitrile
N,N-diallyl-3-methyl-4-nitroaniline
N-(sec-butyl)-4-nitro-N-propyl-3-(trifluoromethyl)aniline
4-(dipentylamino)-2-nitrobenzonitrile
N-(cyclopropylmethyl)-3-methyl-4-nitro-N-propylaniline
5-(dipropylamino)-2-nitrobenzonitrile
N,N-dibutyl-4-nitro-3-(trifluoromethyl)aniline
N1 -(2-chloro-4-nitrophenyl)-N1,N2,N2-trimethyl-1,2-ethanediamine
5-[sec-butyl(propyl)amino]-2-nitrobenzonitrile
N-ethyl-N-(2-methyl-2-propenyl)-4-nitro-3-(trifluoromethyl)aniline
N-(2-methoxyethyl)-4-nitro-N-propyl-3-(trifluoromethyl)aniline
4-[butyl(ethyl)amino]-2-chlorobenzonitrile
5-(dibutylamino)-2-nitrobenzonitrile
4-[butyl(ethyl)amino]-2-nitrobenzonitrile
4-[(2-methoxyethyl)(propyl)amino]-2-(trifluoromethyl)benzonitrile
5-[(2-methoxyethyl)(propyl)amino]-2-nitrobenzonitrile
4-[allyl(cyclopentyl)amino]-2-(trifluoromethyl)benzonitrile
4-[butyl(propyl)amino]-2-(trifluoromethyl)benzonitrile
N,N-dibutyl-3-methyl-4-nitroaniline
4-[methyl(octyl)amino]-2-nitrobenzonitrile
4-(dibutylamino)-2-(trifluoromethyl)benzonitrile
N-(cyclopropylmethyl)-4-nitro-N-propyl-2-(trifluoromethyl)aniline
N-allyl-N-cyclohexyl-4-nitro-3-(trifluoromethyl)aniline
4-[(2-methoxyethyl)(methyl)amino]-3-(trifluoromethyl)benzonitrile
4-(diallylamino)-3-nitrobenzonitrile
N-1-(2-chloro-4-nitrophenyl)-N1,N3,N3-trimethyl-1,3-propanediamine
N,N-bis(2-methoxyethyl)-4-nitro-3-(trifluoromethyl)aniline
4-[sec-butyl(propyl)amino]-2-(trifluoromethyl)benzonitrile
2-chloro-4-[ethyl(2-methyl-2-propenyl)amino]benzonitrile
N-cyclohexyl-N-ethyl-4-nitro-3-(trifluoromethyl)aniline
2-chloro-4-(dibutylamino)benzonitrile
4-[cyclohexyl(ethyl)amino]-2-nitrobenzonitrile
4-[bis(2-ethoxyethyl)amino]-3-chlorobenzonitrile
2-chloro-N-(2-methoxyethyl)-N-methyl-4-nitroaniline
N-butyl-N-ethyl-4-nitro-3-(trifluoromethyl)aniline
N-(sec-butyl)-3-methyl4-nitro-N-propylaniline
N-(2-methoxyethyl)-N-methyl-4-nitro-2-(trifluoromethyl)aniline
4-{bis[3-(dimethylamino)propyl]amino}-2-(trifluoromethyl)benzonitrile
N-(cyclopropylmethyl)-4-nitro-N-propyl-3-(trifluoromethyl)aniline
4-[(methyl)(octyl)amino]-2-(trifluoromethyl)benzonitrile
4-(propylamino)-2-(trifluoromethyl)benzonitrile
4-nitro-N-propyl-3-(trifluoromethyl)aniline
3-{[4-nitro-3-(trifluoromethyl)phenyl]amino}propan-1-ol
4-[(3-hydroxypropyl)amino]-2-(trifluoromethyl)benzonitrile
4-{[2-hydroxy-1-(hydroxymethyl)ethyl]amino}-2-(trifluoromethyl)benzonitrile
4-(dimethylamino)-2-(trifluoromethyl)benzonitrile
4-(diethylamino)-2-(trifluoromethyl)benzonitrile
4-[methyl(2-methylpropyl)amino]-2-(trifluoromethyl)benzonitrile
4-[(cyclopropylmethyl)amino]-2-(trifluoromethyl)benzonitrile
4-[(cyclopropylmethyl)(3-hydroxypropyl)amino]-2-(trifluoromethyl)benzonitrile
4-[(cyclopropylmethyl)(2-hydroxyethyl)amino]-2-(trifluoromethyl)benzonitrile
N-(cyclopropylmethyl)-4-nitro-3-(trifluoromethyl)aniline
3-{(cyclopropylmethyl)[4-nitro-3-(trifluoromethyl)phenyl]amino}-1-propanol
2-{(cyclopropylmethyl)[4-nitro-3-(trifluoromethyl)phenyl]amino}ethanol
4-[(cyclopropylmethyl)amino]-3-(trifluoromethyl)benzonitrile
1-[4-[(cyclopropymethyl)(propyl)amino]-2-(trifluoromethyl)phenyl]ethanone
4-[(1-cyclopropylethyl)amino]-2-(trifluoromethyl)benzonitrile
4-[allyl(1-cyclopropylethyl)amino]-2-(trifluoromethyl)benzonitrile
4-[(2,2-dimethylpropyl)amino]-2-(trifluoromethyl)benzonitrile
4-[(2,2-dimethylpropyl)(3-hydroxypropyl)amino]-2-(trifluoromethyl)benzonitrile
4-[(2,2-dimethylpropyl)(2-propen-1-yl)amino]-2-(trifluoromethyl)benzonitrile
4-[(2,3-dihydroxypropyl)(2,2-dimethylpropyl)amino]-2-(trifluoromethyl)benzonitrile
4-[(2,2-dimethylpropyl)(2-oxoethyl)amino]-2-(trifluoromethyl)benzonitrile
4-[(2,2-dimethylpropyl)(2-hydroxyethyl)amino]-2-(trifluoromethyl)benzonitrile
4-[(2,2-dimethyl propyl)(propyl)amino]-2-(trifluoromethyl)benzon itrile
4-[(1,1-dimethylethyl)amino]-2-(trifluoromethyl)benzonitrile
N-(1,1-dimethylethyl)-4-nitro-3-(trifluoromethyl)aniline
N-(cyclopropylmethyl)-N-(1,1-dimethylethyl)-4-nitro-3-(trifluoromethyl)aniline
4-[(1,1-dimethylethyl)(2-propen-1-yl)amino]-2-(trifluoromethyl)benzonitrile
4-[(1,1-dimethylethyl)(2-oxoethyl)amino]-2-(trifluoromethyl)benzonitrile
4-[(1,1-dimethylethyl)(propyl)amino]-2-(trifluoromethyl)benzonitrile
4-{(3-hydroxypropyl)[(1S)-1-methylpropyl]amino}-2-(trifluoromethyl)benzonitrile
2- (trifluoromethyl)-4-[(3,3,3-trifluoropropyl)amino]benzonitrile
4-[bis(2-fluoroethyl)amino]-2-(trifluoromethyl)benzonitrile
4-[(2,2,2-trifluoroethyl)amino]-2-(trifluoromethyl)benzonitrile
4-nitro-N-(2,2,2-trifluoroethyl)-3-(trifluoromethyl)aniline
4-[(3-hydroxypropyl)(2,2,2-trifluoroethyl)amino]-2-(trifluoromethyl)benzonitrile
4-[(3-hydroxypropyl)(2,2,2-trifluoroethyl)amino]-3-(trifluoromethyl)benzonitrile
4-bromo-N,N-bis(2,2,2-trifluoroethyl)-3-(trifluoromethyl)aniline
4-[bis(2,2,2-trifluoroethyl)amino]-2-(trifluoromethyl)benzonitrile
4-[(2,2-difluoroethyl)(2,2,2-trifluoroethyl)amino]-2-(trifluoromethyl)benzonitrile
bis(2,2,2-trifluoroethyl)[2-(trifluoromethyl)-4-biphenylyl]amine
4-[(2-hydroxyethyl)(2,2,2-trifluoroethyl)amino]-2-(trifluoromethyl)benzonitrile
4-[[2-(methyloxy)ethyl](2,2,2-trfluoroethyl)amino]-2-(trifluoromethyl)benzonitrile
4-[[2-(ethyloxy)ethyl](2,2,2-trifluoroethyl)amino]-2-(trifluoromethyl)benzonitrile
4-((2,2,2-trifluoroethyl){2-[(2,2,2-trifluoroethyl)oxy]ethyl}amino)-2-(trifluoromethyl)benzonitrile
4-[methyl(2,2,2-trifluoroethyl)amino]-2-(trifluoromethyl)benzonitrile
4-[ethyl(2,2,2-trifluoroethyl)amino]-2-(trifluoromethyl)benzonitrile
4-[propyl(2,2,2-trifluoroethyl)amino]-2-(trifluoromethyl)benzonitrile
4-[butyl(2,2,2-trifluoroethyl)amino]-2-(trifluoromethyl)benzonitrile
4-[(2-methylprop-2-enyl)(2,2,2-trifluoroethyl)amino]-2-(trifluoromethyl)benzonitrile
4-[isobutyl(2,2,2-trifluoroethyl)amino]-2-(trifluoromethyl)benzonitrile
4-[(3-methylbut-2-enyl)(2,2,2-trifluoroethyl)amino]-2-(trifluoromethyl)benzonitrile
4-[isopentyl(2,2,2-trifluoroethyl)amino]-2-(trifluoromethyl)benzonitrile
4-[prop-2-ynyl(2,2,2-trifluoroethyl)amino]-2-(trifluoromethyl)benzonitrile
4-[(2-fluoroethyl)(2,2,2-trifluoroethyl)amino]-2-(trifluoromethyl)benzonitrile
4-[[2-(methylthio)ethyl](2,2,2-trifluoroethyl)amino]-2-(trifluoromethyl)benzonitrile
4-[(2-azidoethyl)(2,2,2-trifluoroethyl)amino]-2-(trifluoromethyl)benzonitrile
N-{2-[[4-cyano-3-(trifluoromethyl)phenyl](2,2,2-trifluoroethyl)amino]ethyl}acetamide
methyl 2-[[4-cyano-3-(trifluoromethyl)phenyl](2,2,2-trifluoroethyl)amino]ethylcarbamate
tert-butyl 2-[[4-cyano-3-(trifluoromethyl)phenyl](2,2,2-trifluoroethyl)amino]ethylcarbamate
N-{2-[[4-cyano-3-(trifluoromethyl)phenyl](2,2,2-trifluoroethyl)amino]ethyl} methanesulfonamide; and
4-(dipropylamnino)phthalonitrile.

Additional representative AR modulator compounds, agonists, partial agonists, and antagonists according to the current invention include:
4-nitro-N,N-dipropylaniline
N-(cyclopropylmethyl)-4-nitro-N-propylaniline
4-(dipropylamino)benzonitrile; and
N,N-diallyl-4-nitroaniline.

### ABBREVIATIONS

As used herein the symbols and conventions used in these processes, schemes and examples are consistent with those used in the contemporary scientific literature, for example, the Journal of the American Chemical Society or the Journal of Biological Chemistry. Specifically, the following abbreviations may be used in the examples and throughout the specification:
g (grams); mg (milligrams);
L (liters); mL (milliliters);
μL (microliters); psi (pounds per square inch);
M (molar); mM (millimolar);
Hz (Hertz); MHz (megahertz);
mol (moles); mmol (millimoles);
rt (room temperature); min (minutes);
h (hours); mp (melting point);
TLC (thin layer chromatography); t_{R} (retention time);
RP (reverse phase); PPTS (pyridinium p-toluenesulfonate)
Et₃N (triethylamine); TFA (trifluoroacetic acid);
TFAA (trifluoroacetic anhydride); THF (tetrahydrofuran);
CDCl₃ (deuterated chloroform); CD₃OD (deuterated methanol);
SiO₂ (silica); DMSO (dimethylsulfoxide);
EtOAc (ethyl acetate); atm (atmosphere);
HCl (hydrochloric acid); CHCl₃ (chloroform);
DMF (N,N-dimethylformamide); Ac (acetyl);
Cs₂CO₃ (cesium carbonate); Me (methyl);
Et (ethyl); EtOH (ethanol);
MeOH (methanol); tBu (tert-butyl)
MsCl (methanesulfonyl chloride); NMO (4-methylmorpholine-N-oxide);
PS (polymer supported); NMM (N-methyl morpholine)
NMP (1-methyl-2-pyrrolidinone) DIEA (N, N-diisopropylethylamine)

Unless otherwise indicated, all temperatures are expressed in ° C. (degrees Centigrade). All reactions conducted under an inert atmosphere at room temperature unless otherwise noted.

¹H NMR spectra were recorded on a Varian VXR-300, a Varian Unity-300, a Varian Unity-400 instrument, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), or br (broad). 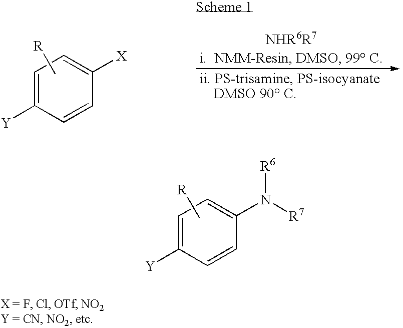
Method A

Compounds of formula (I) can be prepared starting from electron deficient arenes and utilizing solid supported reagents (Scheme 1). The requisite arenes are treated with primary or secondary non-cyclic amines in the presence of a solid supported base such as morpholine to afford the corresponding aniline. Excess halo arene was scavenged with polymer supported trisamine, while excess amine was scavenged with polymer supported isocyanate. 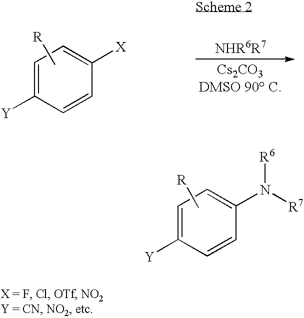
Method B

Compounds of formula (I) can also be prepared starting from electron deficient arenes without the use of polymer supported reagents (Scheme 2). The requisite arenes are treated with primary or secondary non-cyclic amines in the presence of a base such as cesium carbonate to afford the corresponding aniline.

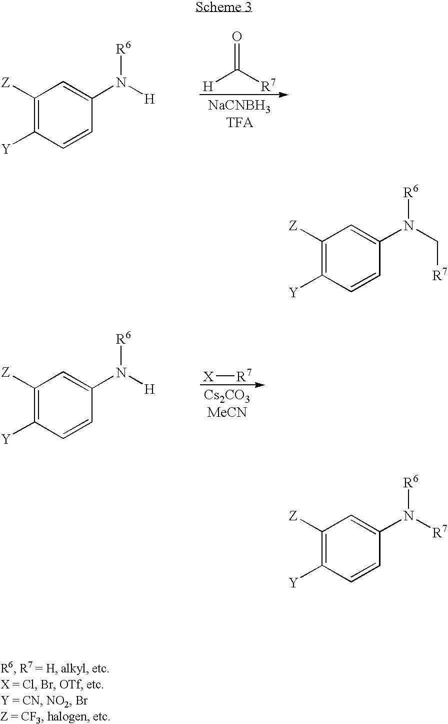

Secondary anilines amenable to the synthesis of compounds of formula (I) can be prepared by two other methods (Scheme 3). Secondary and tertiary anilines are synthesized by reductive alkylation of primary and secondary anilines using aldehydes or hydrates, a non-limiting example of which is trifluoroacetaldehyde hydrate, and reducing agents, a non-limiting example of which is sodium cyanoborohydride, in the presence of acid such as TFA. Another method of secondary and tertiary aniline synthesis involves alkylation of primary and secondary anilines with alkyl halides, a non-limiting example of which is in the presence of base, a non-limiting example of which is sodium hydride.

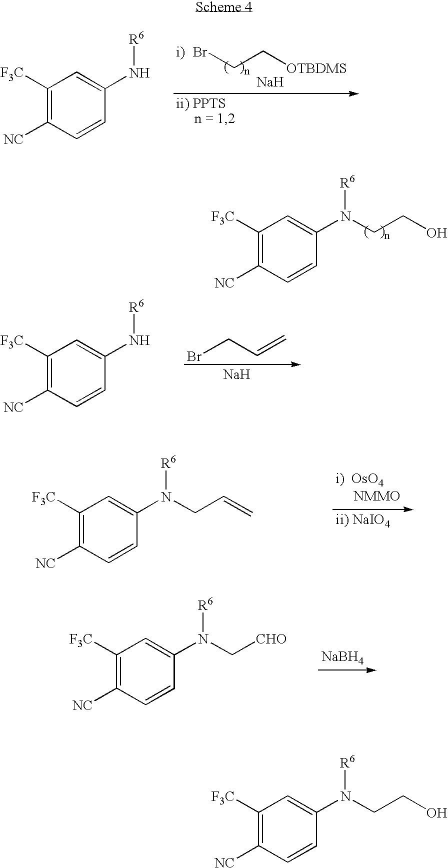

Alcohol bearing tertiary anilines of formula (I) can be prepared by a second alkylation step starting with secondary anilines (Scheme 4). A non-limiting example is alkylation with [(2-bromoethyl)oxy](1,1-dimethylethyl)dimethylsilane in the presence of a base such as sodium hydride. Cleavage of the protecting group affords alcohols. Another method provides alcohols through the oxidative cleavage of olefins and reduction of the corresponding aldehyde with, reagents such as sodium borohydride.

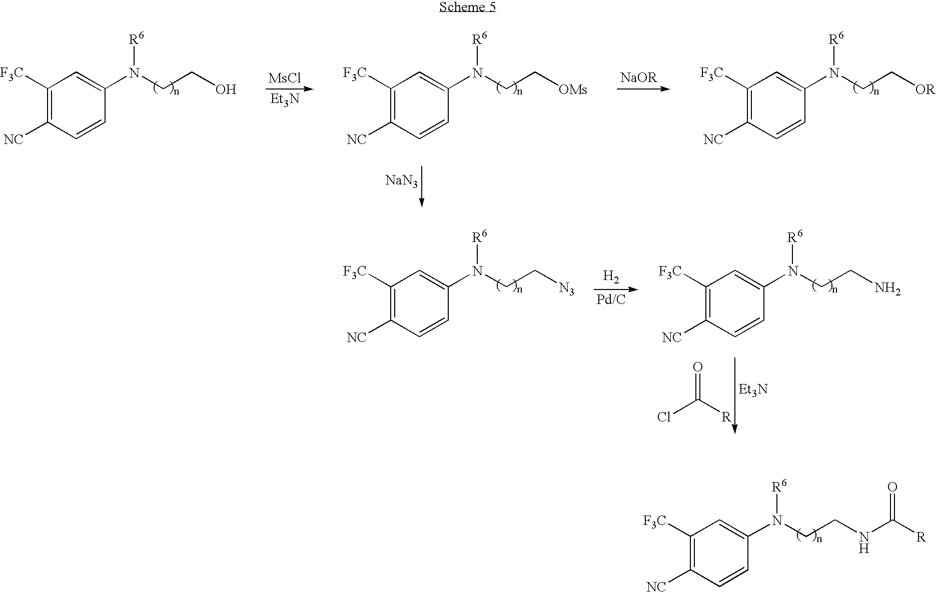

Alcohols of formula (I) can be further elaborated to ethers and amides by conversion to the mesylate (Scheme 5). Treatment of these mesylates with alkoxides affords ethers. Displacement with sodium azide followed by reduction affords the corresponding amine. Amides are formed by treatment of these amines with anhydrides and acid chlorides.

### EXAMPLES

Nucleophilic Aromatic Substitution Method A: Representative Procedure

### EXAMPLE 1

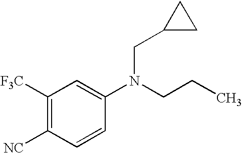 4-[(Cyclopropylmethyl)(propyl)amino]-2-(trifluoromethyl)benzonitrile

Solid supported N-methyl morpholine (PS-NMM) Resin (0.045 g,180 μmol), 120 μL of a 2 M N-(cyclopropylmethyl)-N-propylamine solution in DMSO, and 150 μL of a 1 M 4-fluoro-2-(trifluoromethyl)benzonitrile solution were added to a well of a Robbins FlexChem square well plate. The plate was rotated for 20 h at 99° C. and cooled. To the well was added 1.2 mL of DMSO, solid supported benzylisocyanate (PS-Isocyanate) (0.136 g, 150 μmol), and solid supported trisamine (PS-Trisamine) (0.061 g, 150 μmol). The plate was rotated at 90° C. for 12 h. The solvent in the well was collected via filtration. The resins were rinsed with 0.5 mL DMSO, and the organic solutions were combined and concentrated in vacuo at 60 ° C. to afford an analytically pure white solid (0.034 g, 81%): ¹H NMR (CDCl₃, 400 MHz) δ7.51 (d, J=9.1 Hz, 1H), 7.00 (d, J=2.7Hz, 1H), 6.84 (dd, J=9.0,2.6Hz, 1H), 3.34 (t, J=7.7 Hz, 2H), 3.24 (d, J=6.4 Hz, 2H), 1.63 (sex, J=7.5 Hz, 2H), 1.02 (sept, J=5.4 Hz, 1H), 0.95 (t, J=7.4Hz, 3H), 0.59 (q, J=5.7Hz, 2H), 0.27 (q, J=5.0Hz, 2H).

TABLE 1
All entries in the table below were synthesized according to method A.

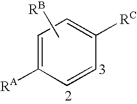

| | | | | | Mass Spec. |
|---|---|---|---|---|---|
| Example | R^{A} | R^{B} | R^{C} | Compound Name | (ES) m/z (M + 1) |
| 2 | CN | 2-NO₂ | 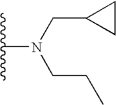 | 4- [(cyclopropylmethyl) (propyl)amino]-2- nitrobenzonitrile | 260 |
| 3 | CN | 2-CF₃ | 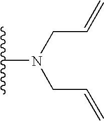 | 4-(diallylamino)-2- (trifluoromethyl)benzo- nitrile | 267 |
| 4 | NO₂ | 2-CN | 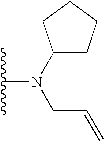 | 5- [allyl(cyclopentyl)amino]-2- nitrobenzonitrile | 272 |
| 5 | CN | 2-NO₂ | 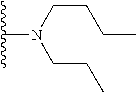 | 4- [butyl(propyl)amino]- 2-nitrobenzonitrile | 262 |
| 6 | CN | 2-NO₂ | 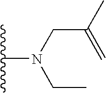 | 4-(ethyl(2-methyl-2- propenyl)amino]-2- nitrobenzonitrile | 246 |
| 7 | CN | 2-CF₃ | 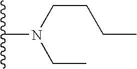 | 4- [butyl(ethyl)amino]- 2- (trifluoromethyl)benzo- nitrile | 271 |
| 8 | CN | 2-CF₃ | 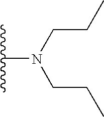 | 4-(dipropylamino)-2- (trifluoromethyl)benzo- nitrile | 271 |
| 9 | NO₂ | 2-CH₃ | 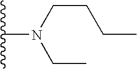 | N-butyl-N-ethyl-3- methyl-4-nitroaniline | 237 |
| 10 | NO₂ | 2-CN | 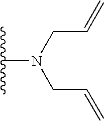 | 5-(diallylamino)-2- nitrobenzonitrile | 244 |
| 11 | NO₂ | 2-CN | 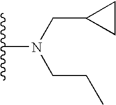 | 5- [cyclopropylmethyl) (propyl)amino]-2- nitrobenzonitrile | 260 |
| 12 | CN | 2-NO₂ | 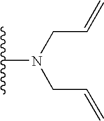 | 4-(diallylamino)-2- nitrobenzonitrile | 244 |
| 13 | NO₂ | 2-CH₃ | 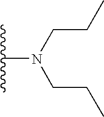 | 3-methyl-4-nitro- N,N-dipropylaniline | 237 |
| 14 | CN | 2-NO₂ | 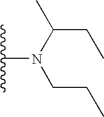 | 4-[sec- butyl(propyl)amino]2-nitrobenzonitrile | 262 |
| 15 | NO₂ | 2-CN | 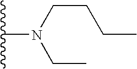 | 5- [butyl(ethyl)amino]- 2-nitrobenzonitrile | 248 |
| 16 | CN | 2-Cl | 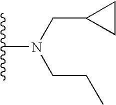 | 2-chloro-4- [(cyclopropylmethyl) (propyl)amino]benzo- nitrile | 249 |
| 17 | NO₂ | 2-CN | 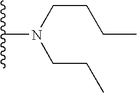 | 5- [butyl(propyl)amino]- 2-nitrobenzonitrile | 262 |
| 18 | NO₂ | 2-CN | 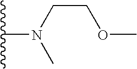 | 5-[(2- methoxyethyl)(methyl) amino]-2- nitrobenzonitrile | 236 |
| 19 | CN | 2-Cl | 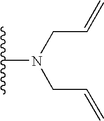 | 2-chloro-4- (diallylamino)benzo- nitrile | 233 |
| 20 | CN | 2-NO₂ | 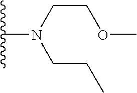 | 4-[(2- methoxyethyl)(propyl) amino]-2- nitrobenzonitrile | 264 |
| 21 | CN | 2-NO₂ | 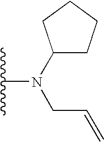 | 4- [allyl(cyclopentyl)amino]-2- nitrobenzonitrile | 272 |
| 22 | CN | 2-CF₃ | 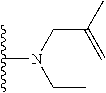 | 4-[ethyl(2-methyl-2- propenyl)amino]-2- (trifluoromethyl)benzo- nitrile | 269 |
| 23 | CN | 2-Cl | 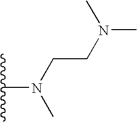 | 2-chloro-4-[[2- (dimethylamino)ethyl](methyl)amino]benzo- nitrile | 238 |
| 24 | NO₂ | 3-CH₃ | 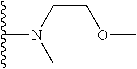 | N-(2-methoxyethyl)- N,2-dimethyl-4- nitroaniline | 225 |
| 25 | NO₂ | 2-CF₃ | 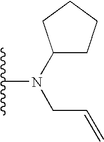 | N-allyl-N- cyclopentyl-4-nitro- 3- (trifluoromethyl)aniline | 315 |
| 26 | NO₂ | 2-OH | 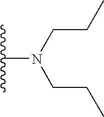 | 5-(dipropylamino)-2- nitrophenol | 239 |
| 27 | CN | 2-Cl | 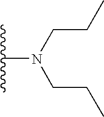 | 2-chloro-4- (dipropylamino)benzo- nitrile | 237 |
| 28 | NO₂ | 2-CN | 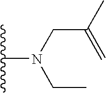 | 5-[ethyl(2-methyl-2- propenyl)amino]-2- nitrobenzonitrile | 246 |
| 29 | NO₂ | 2-CF₃ | 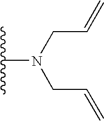 | N,N-diallyl-4-nitro-3- (trifluoromethyl)aniline | 287 |
| 30 | NO₂ | 3-CH₃ | 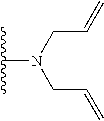 | N,N-diallyl-2-methyl- 4-nitroaniline | 233 |
| 31 | NO₂ | 2-OH | 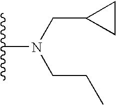 | 5- [(cyclopropylmethyl) (propyl)amino]-2- nitrophenol | 251 |
| 32 | NO₂ | 2-CH₃ | 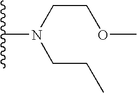 | N-(2-methoxyethyl)- 3-methyl-4-nitro-N- propylaniline | 253 |
| 33 | CN | 2-Cl | 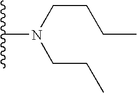 | 4- [butyl(propyl)amino]- 2-chlorobenzonitrile | 251 |
| 34 | NO₂ | 3-Cl | 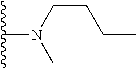 | N-butyl-2-chloro-N- methyl-4-nitroaniline | 242 (M⁺) |
| 35 | CN | 2-NO₂ | 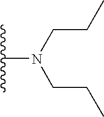 | 4-(dipropylamino)-2- nitrobenzonitrile | 248 |
| 36 | CN | 2-Cl | 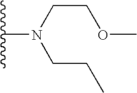 | 2-chloro-4-[(2- methoxyethyl)(propyl) amino]benzonitrile | 253 |
| 37 | NO₂ | 2-CH₃ | 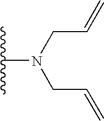 | N,N-diallyl-3-methyl- 4-nitroaniline | 233 |
| 38 | NO₂ | 2-CF₃ | 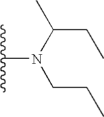 | N-(sec-butyl)-4-nitro- N-propyl-3- (trifluoromethyl)aniline | 305 |
| 39 | CN | 2-NO₂ | 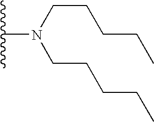 | 4-(dipentylamino)-2- nitrobenzonitrile | 304 |
| 40 | NO₂ | 2-CH₃ | 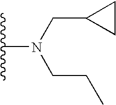 | N- (cyclopropylmethyl)- 3-methyl-4-nitro-N- propylaniline | 249 |
| 41 | NO₂ | 2-CN | 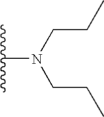 | 5-(dipropylamino)-2- nitrobenzonitrile | 248 |
| 42 | NO₂ | 2-CF₃ | 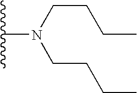 | N,N-dibutyl-4-nitro- 3- (trifluoromethyl)aniline | 319 |
| 43 | NO₂ | 3-Cl | 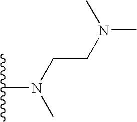 | N¹-(2-chloro-4- nitrophenyl)- N¹,N²,N²-trimethyl- 1,2-ethanediamine | 257 (M⁺) |
| 44 | NO₂ | 2-CN | 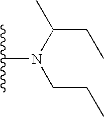 | 5-[sec- butyl(propyl)amino]2-nitrobenzonitrile | 262 |
| 45 | NO₂ | 2-CF₃ | 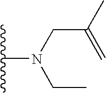 | N-ethyl-N-(2-methyl- 2-propenyl)-4-nitro- (trifluoromethyl)aniline | 289 |
| 46 | NO₂ | 2-CF₃ | 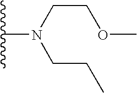 | N-(2-methoxyethyl)- 4-nitro-N-propyl-3- (trifluoromethyl)aniline | 307 |
| 47 | CN | 2-Cl | 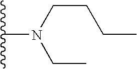 | 4- [butyl(ethyl)amino]- 2-chlorobenzonitrile | 237 |
| 48 | NO₂ | 2-CN | 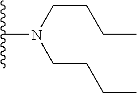 | 5-(dibutylamino)-2- nitrobenzonitrile | 276 |
| 49 | CN | 2-NO₂ | 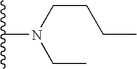 | 4- [butyl(ethyl)amino]2-nitrobenzonitrile | 248 |
| 50 | CN | 2-CF₃ | 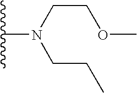 | 4-[(2- methoxyethyl)(propyl) amino]-2- (trifluoromethyl)benzo- nitrile | 287 |
| 51 | NO₂ | 2-CN | 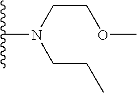 | 5-[(2- methoxyethyl)(propyl) amino]-2- nitrobenzonitrile | 264 |
| 52 | CN | 2-CF₃ | 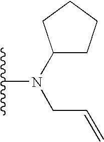 | 4- [allyl(cyclopentyl)amino]-2- (trifluoromethyl)benzo- nitrile | 295 |
| 53 | CN | 2-CF₃ | 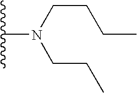 | 4- [butyl(propyl)amino]- 2- (trifluoromethyl)benzo- nitrile | 285 |
| 54 | NO₂ | 2-CH₃ | 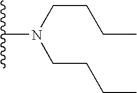 | N,N-dibutyl-3- methyl-4-nitroaniline | 265 |
| 55 | CN | 2-NO₂ | 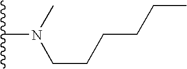 | 4- [methyl(octyl)amino]- 2-nitrobenzonitrile | 290 |
| 56 | CN | 2-CF₃ | 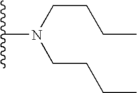 | 4-(dibutylamino)-2- (trifluoromethyl)benzo- nitrile | 299 |
| 57 | NO₂ | 3-CF₃ | 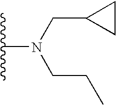 | N- (cyclopropylmethyl) 4-nitro-N-propyl-2- (trifluoromethyl)aniline | 303 |
| 58 | NO₂ | 2-CF₃ | 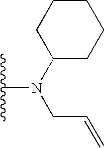 | N-allyl-N-cyclohexyl- 4-nitro-3- (trifuoromethyl)aniline | 329 |
| 59 | CN | 3-CF₃ | 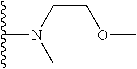 | 4-[(2- methoxyethyl)methyl) amino]-3- (trifluoromethyl)benzo- nitrile | 259 |
| 60 | CN | 3-NO₂ | 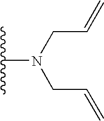 | 4-(diallylamino)-3- nitrobenzonitrile | 244 |
| 61 | NO₂ | 3-Cl | 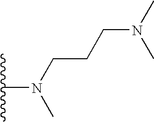 | N¹-(2-chloro-4- nitrophenyl) 1,3-propanediamine | 272 |
| 62 | NO₂ | 2-CF₃ | 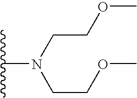 | N,N-bis(2- methoxyethyl)-4- nitro-3- (trifluoromethyl)aniline | 323 |
| 63 | CN | 2-CF₃ | 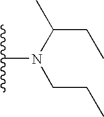 | 4-[sec- butyl(propyl)amino]- 2- (trifluoromethyl)benzo- nitrile | 285 |
| 64 | CN | 2-Cl | 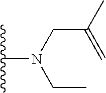 | 2-chloro-4-[ethyl(2- methyl-2- propenyl)amino]benzo- nitrile | 235 |
| 65 | NO₂ | 2-CF₃ | 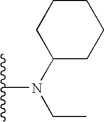 | N-cyclohexyl-N- ethyl-4-nitro-3- (trifluoromethyl)aniline | 317 |
| 66 | CN | 2-Cl | 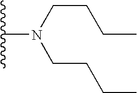 | 2-chloro-4- (dibutylamino)benzo- nitrile | 265 |
| 67 | CN | 2-NO₂ | 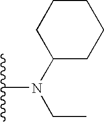 | 4- [cyclohexyl(ethyl)amino]-2- nitrobenzonitrile | 274 |
| 68 | CN | 3-Cl | 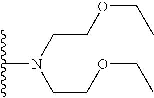 | 4-[bis(2- ethoxyethyl)amino]- 3-chlorobenzonitrile | 297 |
| 69 | NO₂ | 3-Cl | 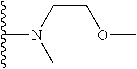 | 2-chloro-N-(2- methoxyethyl)-N- methyl-4-nitroaniline | 245 |
| 70 | NO₂ | 2-CF₃ | 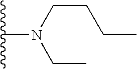 | N-butyl-N-ethyl-4- nitro-3- (trifluoromethyl)aniline | 291 |
| 71 | NO₂ | 2-CH₃ | 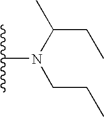 | N-(sec-butyl)-3- methyl-4-nitro-N- propylaniline | 251 |
| 72 | NO₂ | 3-CF₃ | 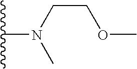 | N-(2-methoxyethyl)- N-methyl-4-nitro-2- (trifluoromethyl)aniline | 279 |
| 73 | NO₂ | H | 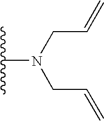 | N,N-diallyl-4- nitroaniline | 219 |
| 74 | NO₂ | H | 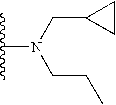 | N- (cyclopropylmethyl)- 4-nitro-N- propylaniline | 235 |
| 75 | CN | H | 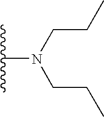 | 4- (dipropylamino)benzo- nitrile | 203 |
| 76 | NO₂ | H | 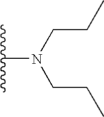 | 4-nitro-N,N- dipropylaniline | 223 |

Nucleophilic Aromatic Substitution Method B: Representative Procedure

### EXAMPLE 1

### Method B

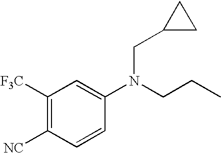 4-[(Cyclopropylmethyl)(propyl)amino]-2-(trifluoromethyl)benzonitrile

A DMSO (0.5 mL) solution of 4-fluoro-2-(trifluoromethyl)benzonitrile (0.050 g, 0.26 mmol, 1 equiv) was treated with cesium carbonate (0.120 g, 0.37 mmol, 1.4 equiv) and N-(cyclopropylmethyl)-N-propylamine (0.035 g, 0.31 mmol, 1.2 equiv). After 3 h at 90° C., the cooled reaction was treated with H₂O (1 mL), and extracted with EtOAc (3×1 mL). Concentration was followed by radial chromatography (SiO₂, 1 mm plate, 90:10; Hex/EtOAc) to afford the title compound as a white solid (0.060 g, 81%): ¹H NMR (CDCl₃, 400 MHz) δ7.51 (d, J=9.1 Hz, 1 H), 7.00 (d, J=2.7 Hz,1 H), 6.84 (dd, J=9.0, 2.6 Hz, 1H), 3.34 (t, J=7.7 Hz, 2H), 3.24 (d, J=6.4 Hz, 2H), 1.63 (sex, J=7.5 Hz, 2H), 1.02 (sept, J=5.4 Hz, 1 H), 0.95 (t, J=7.4 Hz, 3H), 0.59 (q, J=5.7 Hz, 2H), 0.27 (q, J=5.0 Hz, 2H).

### EXAMPLE 77

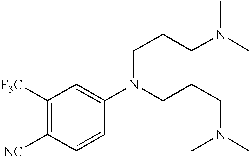 4{bis[3-(Dimethylamino)propyl]amino}-2-(trifluoromethyl)benzonitrile

Synthesized according to example 1, method B using bis[3-(dimethylamino)propyl]amine: ¹H NMR (DMSO d6, 400 MHz) 67 7.79 (d, J=9.5 Hz, 1 H), 7.06-7.04 (m, 2H), 3.46 (t, J=7.2 Hz, 4H), 2.93 (bs, 4H), 2.66 (s, 12H), 1.84-1.79 (m, 4H).

### EXAMPLE 78

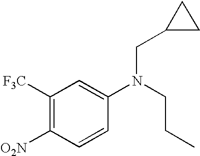 N-(Cyclopropylmethyl)-4-nitro-N-propyl-3-(trifluoromethyl)aniline

Synthesized according to example 1, method B using 4-fluoro-1-nitro-2-(trifluoromethyl)benzene: ¹H NMR (CDCl₃, 400 MHz) δ8.06 (d, J=9.4 Hz, 1H), 6.99 (d, J=2.6 Hz, 1H), 6.74 (dd, J=9.5, 3.0 Hz, 1 H), 3.42 (t, J=7.7 Hz, 2H), 3.31 (d, J=6.4 Hz, 2H), 1.67 (sex, J=7.4 Hz, 2H), 1.04 (sept, J=6.0 Hz, 1 H), 0.95 (t, J=7.4 Hz, 3H), 0.62 (q, J=5.6 Hz, 2H), 0.29 (q, J=5.1 Hz, 2H).

### EXAMPLE 79

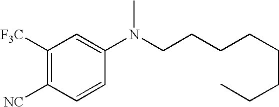 4-[(Meihyl)(octyl)amino]-2-(trifluoromethyl)benzonitrile

Synthesized according to example 1, method B using N-methyl-N-octylamine: ¹H NMR (CDCl₃, 400 MHz) δ7.55 (d, J=8.8 Hz, 1 H), 6.87 (d, J=2.8 Hz, 1 H), 6.71 (dd, J=8.8, 2.8 Hz,1 H), 3.39 (t, J=7.2 Hz, 2H), 3.44 (s, 3H), 1.62-1.55 (m, 2H), 1.31-1.23 (m, 10 H), 0.88 (t, J=6.8 Hz, 3H); MS (APCl) m/z313 (M+1).

### EXAMPLE 80

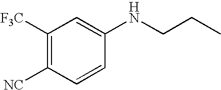 4-(Propylamino)-2-(trifluoromethyl)benzonitrile

Synthesized according to example 1, method B using 1-aminopropane: ¹H NMR (CDCl₃) 400 MHz δ7.54 (d, J=8.6 Hz, 1 H), 6.83 (d, J=2.2 Hz, 1 H), 6.66 (dd, J=8.6, 2.2 Hz,₁ H), 4.43 (bs, 1H), 3.18-3.13 (m, 2H), 1.72-1.60 (m, 2H), 1.01 (t, J=7.5 Hz, 3H); MS (ES) mlz 227 (M-1).

### EXAMPLE 81

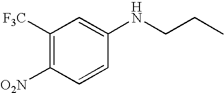 4-Nitro-N-propyl-3-(trifluoromethyl)aniline

Synthesized in a manner similar to example 1, method B using 4-fluoro-1-nitro-2-(trifluoromethyl)benzene and 1-aminopropane: MS (ES) mlz 249 (M+1).

### EXAMPLE 82

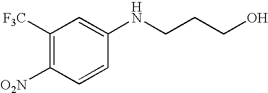 3-{[4-Nitro-3-(trifluoromethyl)phenyl]amino}propan-1-ol

Synthesized in a manner similar to example 1, method B using using 4-fluoro-1 -nitro-2-(trifluoromethyl)benzene and 3-aminopropan-1-ol: MS (ES) mlz 263 (M−1).

### EXAMPLE 83

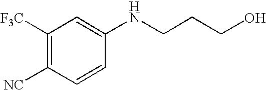 4-[(3-Hydroxypropyl)amino]-2-(trifluoromethyl)benzonitrile

Synthesized in a manner similar to example 1, method B using 3-aminopropan-1-ol: MS (ES) mlz 243 (M−1).

### EXAMPLE 84

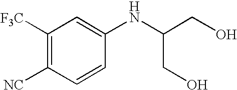 4-{[2-Hydroxy-1 -(hydroxymethyl)ethyl]amino}-2-(trifluoromethyl)benzonitrile

Synthesized in a manner similar to example 1, method B using 2-amino-1,3-propanediol: MS (ES) mlz 261 (M+1).

### EXAMPLE 85

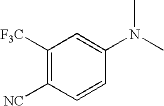 4-(Dimethylamino)-2-(trifluoromethyl)benzonitrile

Synthesized in a manner similar to example 1, method B using dimethylamine: MS (ES) mlz 215 (M+1).

### EXAMPLE 86

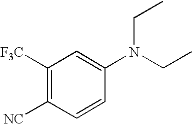 4-(Diethylamino)-2-(trifluoromethyl)benzonitrile

A solution of 4-fluoro-2-(trifluoromethyl)benzonitrile (0.050 g, 0.264 mmol) and diethylamine (0.096 g, 1.32 mmol) in acetonitrile (3 mL) was heated in a microwave at 120° C. for 10 min. Upon cooling, additional diethylamine (0.096 g, 1.32 mmol) was added and the reaction was heated in a microwave at 150° C. for 20 min. Upon cooling, the reaction mixture was partitioned between EtOAc and water. The organic phase was washed with water and brine, dried (Na₂SO₄) and concentrated in vacuo. The residue was purified by silica gel chromatography (5-40% EtOAc-hexane gradient) to give the title compound (0.058 g, 90% yield): MS (ES) mlz 243 (M+1).

### EXAMPLE 87

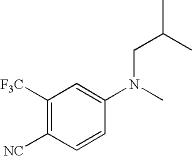 4-[Methyl(2-methylpropyl)aminol-2-(trifluoromethyl)benzonitrile

Synthesized in a manner similar to example 1, method B using methyl(2-methylpropyl)amine: ¹H NMR (400 MHz, CDCl₃) δ7.55 (d, J=8.8 Hz, 1 H), 6.87 (d, J=2.7 Hz, 1H), 6.71 (dd, J=8.8, 2.7 Hz, 1 H), 3.21 (d, J=7.4 Hz, 2H), 3.06 (s, 3H), 2.05 (m, 1H), 0.93 (d, J=6.8 Hz, 6H).

### EXAMPLE 88

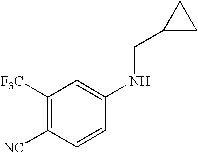 4-[(Cyclopropylmethyl)aminol-2-(trifluoromethyl)benzonitrile

Synthesized in a manner similar to example 1, method B using (cyclopropylmethyl)amine: ¹H NMR (400 MHz, CDCl₃) δ7.53 (d, J=8.6 Hz, 1 H), 6.83 (d, J=2.4 Hz, 1H), 6.65 (dd, J=8.6, 2.4 Hz, 1H), 4.62 (bs, 1H), 3.02 (d, J=7.1 Hz, 2H), 1.09 (m, 1H), 0.61 (m, 2H), 0.27 (m, 2H).

### EXAMPLE 89

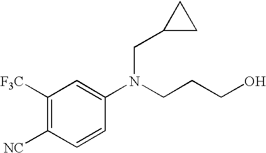 4-[(Cyclopropyimethyl)(3-hydroxypropyl)amino]-2-(trifluoramethyl)benzonitrile

To a slurry of hexanes-washed NaH (0.144 g of a 60% w/w suspension in mineral oil, 3.59 mmol) in dry DMF (5 mL) at 0° C. was added a solution of example 88 (0.345 g, 1.44 mmol) in DMF (2 mL), dropwise over 3 min. The mixture was stirred 30 min and [(3-bromopropyl)oxy](1,1 -dimethylethyl)dimethylsilane (0.67 mL, 2.9 mmol) was added via syringe. The cooling bath was removed and the mixture was stirred at room temp. After 2 h, the mixture was poured into water and the whole was extracted with Et₂O (×3). Combined organic portions were washed (water, brine), dried over Na₂SO₄, filtered, and concentrated in vacuo. The residue was purified by flash chromatography (EtOAc/hexanes), affording 0.499 g of the intermediate TBS ether as a colorless oil. ¹H NMR (300 MHz, CDCl₃) δ7.55 (d, J=8.8 Hz,1H), 6.99 (d, J=2.3 Hz, 1 H), 6.86 (dd, J=8.9, 2.4 Hz, 1H), 3.66 (t, J=5.5 Hz, 2H), 3.57 (t, J=7.4 Hz, 2H), 3.29 (d, J=6.2 Hz, 2H), 1.73-1.86 (m, 2H), 0.97-1.11 (m,1 H), 0.92 (s, 9H), 0.56-0.65 (m, 2H), 0.24-0.32 (m, 2H), 0.07 (s, 6H). A mixture of the silyl ether (0.499 g) and PPTS (0.200 g) in EtOH (10 mL) was brought to reflux. After 3 h, the mixture was cooled and concentrated in vacuo. The residue was partitioned between EtOAc/satd NaHCO₃ and the layers were separated. The organic layer was washed (10% HCl, water, brine), dried over Na₂SO₄, filtered, and concentrated in vacuo. The residue was purified by flash chromatography (EtOAc/hexanes), affording 0.302 g of the title compound as a colorless oil which slowly solidified to a waxy solid: ¹H NMR (300 MHz, MeOH-d₄) δ7.63 (d, J=8.7 Hz, 1H), 7.11 (d, J=2.6 Hz, 1H), 7.02 (dd, J=9.0, 2.7 Hz, 1H), 3.66-3.56 (m, 4H), 3.37 (d, J=6.4 Hz, 2H), 1.83 (tt, J=7.2, 6.2 Hz, 2H), 1.13-0.98 (m, 1H), 0.64-0.53 (m, 2H), 0.37-0.28 (m, 2H).

### EXAMPLE 90

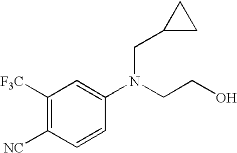 4-[(Cyclopropylmethyl)(2-hydroxyethyl)amino]-2-(trifluoromethyl)benzonitrile

Synthesized as described in example 89 from example 88 and [(2-bromoethyl)oxy](1,1-dimethylethyl)dimethylsilane: ¹H NMR (300 MHz, CDCl₃) δ7.55 (d, J=8.8 Hz, 1H), 7.05 (d, J=2.3 Hz, 1H), 6.89 (dd, J=8.8, 2.6 Hz, 1H), 3.87 (bt, J =5.7 Hz, 2H), 3.67 (t, J=5.8 Hz, 2H), 3.36 (d, J=6.0 Hz, 2H), 1.78 (bs, 1H), 1.12-0.97 (m, 1H), 0.69-0.56 (m, 2H), 0.35-0.25 (m, 2H); MS (APCI) mlz285 (M+1).

### EXAMPLE 91

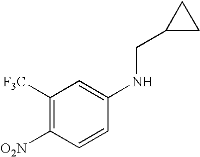 N-(Cyclopropylmethyl)-4-nitro-3-(trifluoromethyl)aniline

Synthesized as described in example 1, method B from 4-fluoro-1-nitro-2-(trifluoromethyl)benzene and (cyclopropylmethyl)amine: MS (APCI) mlz 261 (M+1).

### EXAMPLE 92

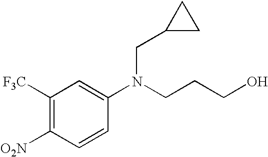 3-{(Cyclopropylmethyl)[4-nitro-3-(trifluoromethyl)phenyl]amino}-1-propanol

Synthesized as described in example 89 from N-(cyclopropylmethyl)-4-nitro-3-(trifluoromethyl)aniline and [(3-bromopropyl)oxy](1,1-dimethylethyl)dimethylsilane: MS (APCI) mlz 319 (M+1).

### EXAMPLE 93

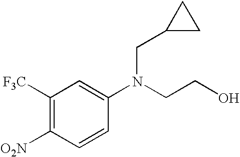 2-{(Cyclopropylmethyl)[4-nitro-3-(trifluoromethyl)phenyl]amino}ethanol

Synthesized as described in example 89 from N-(cyclopropylmethyl)- 4-nitro-3-(trifluoromethyl)aniline and [(2-bromoethyl) oxy](1,1 -dimethylethyl)dimethylsilane: ¹H NMR (300 MHz, CDCl₃) δ8.03 (d, J=9.4 Hz, 1H), 7.10 (d, J=2.6 Hz, 1H), 6.86 (dd, J=9.5, 2.6 Hz, 1H), 3.90 (bt, J=5.6 Hz, 2H), 3.71 (t, J=5.8 Hz, 2H), 3.40 (d, J6.3 Hz, 2H), 1.71 (bs, 1H), 1.14-0.99 (m, 1H), 0.64 (overlapping td, 2H), 0.32 (overlapping td, 2H).

### EXAMPLE 94

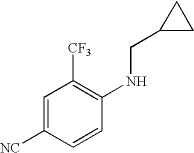 4-[(Cyclopropylmethyl)amino]-3-(trifluoromethyl)benzonitrile

Synthesized as described in example 1, method B from 4-fluoro-3-(trifluoromethyl)benzonitrile and (cyclopropylmethyl)amine: MS (APCI) mlz 241 (M+1).

### EXAMPLE 95

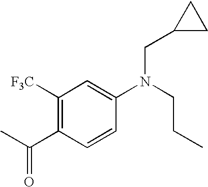 1-[4-[(Cyclopropylmethyl)(propyi)amino]-2-(trifluoromethyl)phenyl]ethanone

Synthesized in a manner similar to example 1, method B using 4-fluoro-2-trifluoromethylacetophenone and (cyclopropylmethyl)propylamine: ¹H NMR (CDCl₃, 400 MHz) δ7.54 (d, J=9.0 Hz, 1H), 6.93 (d, J=2.3 Hz, 1H), 6.76 (dd, J=8.9, 2.3 Hz, 1 H), 3.37 (t, J=7.8 Hz, 2H), 3.28 (d, J=6.4 Hz, 2H), 2.54 (s, 3H), 1.70-1.60 (m, 2H), 1.08-1.00 (m, 1 H), 0.95 (t, J=7.5 Hz, 3H), 0.61-0.55 (m, 2H), 0.30-0.25 (m, 2H); MS (APCI) mlz 300 (M+1).

### EXAMPLE 96

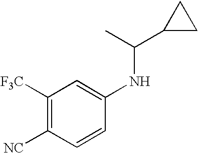 4-[(1 -Cyclopropylethyl)amino]-2-(trifluoromethyl)benzonitrile

Synthesized in a manner similar to example 1, method B from 4-fluoro-2-(trifluoromethyl)benzonitrile and 1-cyclopropylethyl amine (ref. J. L. Kelly, R. M. Morris, M. P. Krochmal, E. W. McLean, J. A. Linn, M. J. Durcan, D. R. Cooper, J. Med. Chem., 1997, 40(20), 3207-3216): ¹H NMR (CDCl₃ 400 MHz) δ7.50 (d, J=8.6Hz, 1H), 6.80 (d, J=2.2 Hz, 1H), 6.63 (dd, J=8.6, 2.2 Hz, 1H), 4.57 (bd, J=6.4Hz, 1H), 3.08-3.01 (m, 1 H), 1.25 (d, J=6.4 Hz, 3H), 0.95-0.86 (m, 1H), 0.61-0.55 (m, 2H), 0.30-0.25 (m, 2H).

### EXAMPLE 97

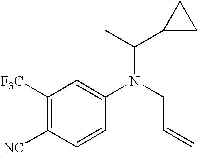 4-[Allyl(1-cyclopropylethyl)aminol-2-(trifluoromethyl)benzonitrile

A DMF solution of example 96 (0.040 g, 0.157 mmol) was cooled to 0° C. under N₂ with stirring. NaH (0.011 g, 0.472 mmol) was added slowly with gas evolution and the reaction mixture stirred for 30 min. Allyl bromide (0.020 mL, 0.236 mmol) was added and the reaction was warmed to ambient temperature and stirred for 18 h. H₂O was added and the organic portions were extracted with EtOAc. The organic portions were washed with brine and then dried, filtered, and concentrated to an oily residue which was subjected to chromatography on silica gel using hexanes:EtOAc to afford the title compound as a pale yellow oil (0.043g, 93% yield): ¹H NMR (CDCl₃, 400 MHz) δ7.52 (d, J=9.0 Hz, 1H), 6.93 (d, J=2.2 Hz, 1H), 6.75 (dd, J=9.0, 2.6 Hz, 1H), 5.89-5.80 (m, 1H), 5.25-5.19 (m, 2H), 4.14-4.09 (m, 1H), 4.02-3.96 (m, 1H), 3.33-3.24 (m, 1H), 1.25 (d, J=6.6 Hz, 3H), 1.03-0.98 (m, 1H), 0.70-0.66 (m, 1H), 0.54-0.50 (m, 1H), 0.37-0.33 (m, 1H), 0.26-0.22 (m,1H).

### EXAMPLE 98

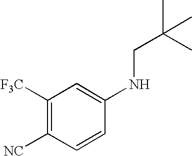 4-1(2,2-Dimethylpropyl)amino]-2-(trifluoromethyl)benzonitrile

Synthesized as described in example 1, method B from 4-fluoro-2-(trifluoromethyl)benzonitrile and neopentylamine: MS (EI) mlz 256 (M⁺, 9%), 239 ({[M—H]—CH₃}⁺, 32%),199 ([M-^{t}butyl]⁺, 78%),170 ([M-neopentylamine]⁺, 100).

### EXAMPLE 99

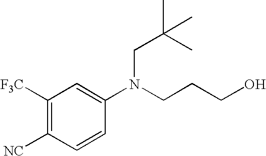 4-[(2,2-Dimethylpropyl)(3-hydroxypropyl)amino]-2-(trifluoromethyl)benzonitrile

Synthesized as described in example 89 from 4-[(2,2-dimethylpropyl)amino]-2-(trifluoromethyl)benzonitrile and [(3-bromopropyl)oxy](1,1-dimethylethyl)dimethylsilane: ¹H NMR (300 MHz, CDCl₃) δ7.53 (d, J=8.9 Hz, 1H), 7.05 (d, J2.4 Hz, 1H), 6.87 (dd, J=8.9, 2.4 Hz, 1H), 3.70 (overlapping td, 2H), 3.62 (t, J=7.3 Hz, 2H), 3.27 (s, 2H), 1.80 (overlapping tt, 2H), 1.47 (br. t, J=4.7 Hz, 1H), 0.99 (s, 9H).

### EXAMPLE 100

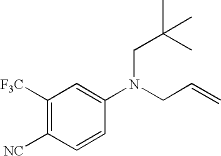 4-[(2,2-Dimethylpropyl)(2-propen-1-yl)amino]-2-(trifluoromethyl)benzonitrile

Synthesized in a manner similar to example 97 from example 98 and allyl bromide: MS (EI) mlz 296 (M⁺, 9%), 239 ([M—^{t}Bu]⁺, 100%).

### EXAMPLE 101

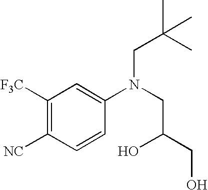 4-[(2,3-Dihydroxypropyl)(2,2-dimethylpropyl)amino]-2-(trifluoromethyl)benzonitrile

To a solution of example 100 (0.0481 g, 0.163 mmol) in THF/water (20:1, 1.6 mL) at room temp. was added a solution of OSO₄ in t-BuOH (0.083 mg of a 2.5% w/w solution, 0.0081 mmol), and NMMO (0.040 g, 0.342 mmol). The mixture was stirred 15 h and sodium bisulfite (0.020 g) was added. The mixture was stirred 1 h, poured into water, and the whole was extracted with EtOAc (×3). Combined organics were washed (water, brine), dried over Na₂SO₄, filtered, and concentrated in vacuo. The residue was purified by flash chromatography (EtOAc/hexanes), affording 0.0465 g of the title compound as a colorless film: MS (APCI) mlz 331 (M+1).

### EXAMPLE 102

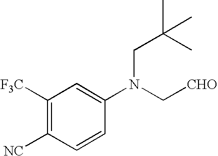 4-1(2,2-Dimethylpropyl)(2-oxoethyl)amino]-2-(trifluoromethyl)benzonitrile

To a solution of example 101 (0.0465 g, 0.141 mmol) in acetone (3 mL) at room temp. was added a solution of NaIO₄ (0.0612 g, 0.286 mmol) in water (1 mL). After 15 h the mixture was poured into water and the whole was extracted with Et₂O (×3). Combined organics were washed (10% Na₂S₂O₃, water, brine), dried over Na₂SO₄, filtered, and concentrated in vacuo. The residue was purified by flash chromatography (EtOAc/hexanes), affording 0.032 g of the title compound as a colorless gum that slowly solidified: ¹H NMR (400 MHz, CDCl₃) δ9.70 (s,1H), 7.57 (d, J=8.8 Hz, IH), 6.96 (d, J=2.5 Hz, 1H), 6.74 (dd, J=8.9, 2.7 Hz, 1 H), 4.31 (s, 2H), 3,33 (s, 2H), 1.02 (s, 9H).

### EXAMPLE 103

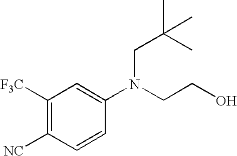 4-[(2,2-Dimethylpropyl)(2-hydroxyethyl)amino]-2-(trifluoromethyl)benzonitrile

To a solution of example 102 (0.882 g, 2.96 mmol) in MeOH (10 mL) at 0° C. was added NaBH₄ (0.112 g, 2.96 mmol) in one portion and the mixture was stirred overnight, slowly warming to room temp. The mixture was cooled to 0° C., satd NH₄CI (1 mL) was added and the mixture was concentrated in vacuo. The residue was partitioned between EtOAc/water and the layers were separated. The organic layer was washed with brine, dried over Na₂SO₄, filtered, and concentrated in vacuo. The residue was purified by flash chromatography (EtOAc/hexanes) affording 0.829 g of the title compound as a colorless syrup that solidified on standing: MS (ES) mlz 301 (M+1).

### EXAMPLE 104

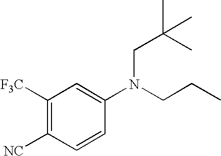 4-[(2,2-Dimethylpropyl)(propyl)amino]-2-(trifluoromethyi)benzonitrile

Synthesized in a manner similar to example 97 from example 98 and 1 -iodopropane: MS (APCI) mlz 299 (M+1).

### EXAMPLE 105

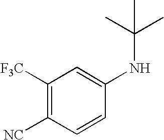 4-1(1,1-Dimethylethyl)amino]-2-(trifluoromethyl)benzonitrile

Synthesized as described in example 1, method B from 4-fluoro-2-(trifluoromethyl)benzonitrile and tert-butylamine: MS (EI) mlz 242 (M⁺, 7%), 227 ([M—CH₃]+, 33%), 211 ({[M—H]- 2CH₃}⁺, 100%), 186 ([M−C₄H₉]⁺, 18%).

### EXAMPLE 106

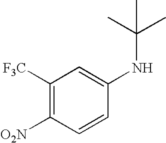 N-(1,I-Dimethylethyl)-4-nitro-3-(trifluoromethyl)aniline

Synthesized as described in example 1, method B from 4-fluoro-1 -nitro-2-(trifluoromethyl)benzene and tert-butylamine: MS (APCI) mlz 263 ([M+H]+, 63%), 207 ({[M+H] —C₄H₈}+, 100%).

### EXAMPLE 107

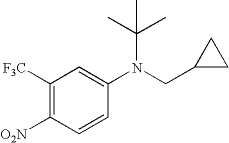 N-(Cyclopropylmethyl)-N-(1,1-dimethylethyl)-4-nitro-3-(trifluoromethyl)aniline

Synthesized in a manner similar to example 97 from example 106 and (bromomethyl)cyclopropane: MS (EI) mlz316 (M⁺, 10%), 301 ([M—CH₃]⁺, 35%), 260 ([M—C₄H₈]⁺, 41%), 55 (100%).

### EXAMPLE 108

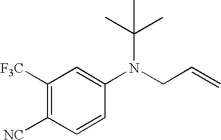 4-[(1,1 -Dimethylethyl)(2-propen-1 -yl)amino]-2-(trifluoromethyl)benzonitrile

Synthesized as described in example 97 from example 105 and allyl bromide: ¹H NMR (400 MHz, CDCI₃) δ7.55 (d, J=8.7 Hz, 1 H), 7.28 (d, J=2.4 Hz, 1H), 7.11 (dd, J=8.7, 2.4 Hz, 1H), 5.87 (ddt, J=17.1, 10.5, 4.2 Hz, 1H), 5.23-5.14 (m, 2H), 4.00 (overlapping ddd, 2H), 1.46 (s, 9H).

### EXAMPLE 109

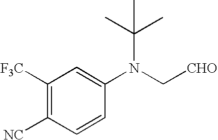 4-[(1,1 -Dimethylethyl)(2-oxoethyl)amino]-2-(trifluoromethyl)benzonitrile

To a solution 4-[(1,-dimethylethyl)(2-propen-1-yl)amino]-2-(trifluoromethyl)benzonitrile (0.911 g, 3.23 mmol) in THF/water (20:1, 30 mL) at room temp. was added a solution of OSO₄ in t-BuOH (0.820 g of a 2.5% wlw solution, 0.081 mmol) and NMMO (0.794 g, 6.78 mmol). The mixture was stirred 20 h at room temp., cooled to 0° C. and sodium bisulfite (0.250 g) was added. The mixture was stirred 1 h at room temp., and the supernatant was carefully decanted away from the gummy residue which had formed. The residue was rinsed with EtOAc (×2), and combined organics were concentrated in vacuo. The residue was partitioned between EtOAc/water and the layers were separated (required addition of NaCI). The aqueous layer was extracted with EtOAc (×2), combined organics were washed (10% HCI, water, brine) and concentrated in vacuo. The residue was dissolved in acetone (30 mL), and to the resulting solution was added a solution of NaIO4 (1.45 g, 6.78 mmol) in water (12 mL). The mixture was stirred 45 min and concentrated in vacuo to an aqueous residue. The residue was extracted with EtOAc (×3), combined organics were washed (water, brine), dried over Na₂SO₄, filtered, and concentrated in vacuo. The residue was purified by flash chromatography (EtOAc/hexanes), affording 0.720 g of the title compound as a pale yellow solid: MS (APCI) mlz 283 ([M—H]^{—}, 27%), 227 ({[M—H]—C₄H₈}^{—}, 100%), 226 ({[M—H]—C₄H₉}^{—}, 83%).

### EXAMPLE 110

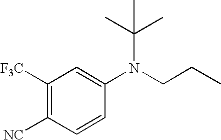 4-[(1,1 -Dimethylethyl)(propyl)amino]-2-(trifluoromethyl)benzonitrile

A slurry of 5% w/w Pd/C (0.022 g, 0.01 mmol Pd) in EtOAc (1 mL) was stirred under an atmosphere of hydrogen for 10 min and a solution example 108 (0.059 g, 0.21 mmol) in EtOAc (1 mL) was added via syringe. The mixture was stirred under hydrogen for 45 min and filtered through Celite. The filtrate was adsorbed onto a small amount of silica gel and purified by flash chromatography (EtOAc/hexanes), affording 0.037 g of the title compound as a colorless, waxy solid: ¹H NMR (400 MHz, CDCI₃) δ7.62 (d, J=8.7Hz, 1H), 7.32 (d, J=2.2Hz, 1H), 7.18 (dd, J=8.7, 2.3 Hz, 1H), 3.23 (t, J=7.6 Hz, 2H), 1.43 (overlapping qt, 2H), 1.33 (s, 9H), 0.88 (t, J =7.3 Hz, 3H).

### EXAMPLE 111

4-{(3-Hydroxypropyl)[(1S)-1 -methylpropyl]amino}-2-(trifluoromethyl)benzonitrile 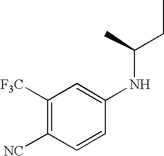
A. 4-{[(1S)-1-Methylpropyl]amino}-2-(trifluoromethyl)benzonitrile

Synthesized as described in example 1, method B using 4-fluoro-2-(trifluoromethyl)benzonitrile and (2S)-2-butanamine: ¹H NMR (300 MHz, CDCI₃) δ7.53 (d, J=8.6 Hz, 1H), 6.82 (d, J=2.2 Hz, 1H), 6.65 (dd, J=2.3 Hz, 1H), 4.38 (bd, J=7.3 Hz, 1H), 3.56-3.40 (m, 1H), 1.69-1.47 (m, 2H), 1.22 (d, J=6.4 Hz, 3H), 0.97 (t, J=7.4 Hz, 3H). 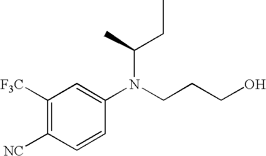
B. 4-{(3-Hydroxypropyl)[(1S)-1-methylpropyl]amino}-2-(trifluoromethyl)benzonitrile

Synthesized as described in example 89 from example 111A and [(3-bromopropyl)oxy](1,1-dimethylethyl)dimethylsilane: ¹H NMR (300 MHz, CDCI₃) δ7.57 (d, J=8.9 Hz, 1H), 7.07 (d, J=2.4 Hz, 1H), 6.88 (dd, J=9.0, 2.6 Hz, 1H), 3.89 (overlapping qt, 1H), 3.78 (bt, J=5.6 Hz, 2H), 3.52-3.35 (m, 2H), 1.94-1.76 (m, 2H), 1.76-1.57 (m, 2H), 1.51 (bs, 1H), 1.27 (d, J=6.7 Hz, 3H), 0.92 (t, J=7.4 Hz, 3H).

### EXAMPLE 112

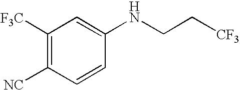 2-(Trifluoromethyl)-4-[(3,3,3-trifluoropropyl)amino]benzonitrile

A mixture of 4fluoro-2-(trifluoromethyl)benzonitrile (0.158 g, 0.84 mmol), 3,3,3-trifluoropropylamine.HCI (0.125 g, 0.84 mmol) and DIEA (0.326 g, 2.52 mmol) in DMSO (1.5 mL) was heated under nitrogen in a microwave at 200° C. for 20 min. The mixture was partitioned between Et₂O and 0.1 N HCI. The organic phase was washed with 0.1 N HCI (twice) and brine, dried (Na₂SO₄), and concentrated in vacuo. The residue was purified by silica gel chromatography (0-50% EtOAc-hexane gradient) and the product crystallized from Et₂O-hexanes to give the title compound as a white solid (0.144 g, 61% yield): ¹H NMR (400 MHz, CDCI₃) δ7.60 (d, J=8.6 Hz, 1H), 6.87 (d, J=2.4 Hz, 1H), 6.71 (dd, J=8.6, 2.4 Hz, 1 H), 4.56 (bs, NH), 3.53 (q, J=6.5 Hz, 2H), 2.51-2.40 (m, 2H); MS (ES) mlz 283 (M+1).

### EXAMPLE 113

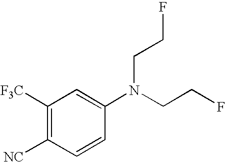 4-[bis(2-Fluoroethyl)amino]-2-(trifluoromethyl)benzon itrile

A mixture of 4-amino-2-(trifluoromethyl)benzonitrile (0.100 g, 0.538 mmol), cesium carbonate (0.438 g, 1.35 mmol) and 1-bromo-2-fluoroethane (0.340 g, 2.68 mmol) in MeCN (3 mL) was heated in a microwave at 120° C. under nitrogen for 15 min. Upon cooling, additional 1-bromo-2-fluoroethane (0.340 g, 2.68 mmol) was added and the mixture was heated in a microwave at 140° C. for 20 min. Upon cooling, the mixture was transferred to a pressure tube, additional 1-bromo-2-fluoroethane (1.00 g, 7.88 mmol) and cesium carbonate (0.208 g, 0.64 mmol) were added and the mixture was heated at 140° C. for 8 h. The reaction was monitored by TLC and additional 1-bromo-2-fluoroethane added accordingly. When approximately 1:1 mixture of mono- and bis-alkylated products was observed by TLC (developed using 70% CH₂CI₂-hexane), the reaction was stopped. The mixture was partitioned between ethyl acetate and water, the organic phase was washed with brine, dried (Na₂SO₄) and concentrated in vacuo. The residue was purified by silica gel chromatography (10-80% dichloromethane-hexane gradient) and the product was crystallized from dichloromethane-hexanes to give the title compound as a white solid (0.028 g, 18% yield): ¹H NMR (400 MHz, CDCI₃) δ7.61 (d, J=8.8 Hz, I H), 6.97 (d, J=2.6 Hz, 1H), 6.84 (dd, J=8.8, 2.6 Hz, 1H), 4.65 (dt, J=46.9, 4.9 Hz, 4H), 3.82 (dt, J=24.7, 4.9 Hz, 4H); MS (ES) mlz 279 (M+1).

### EXAMPLE 114

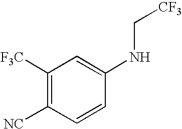 4-[(2,2,2-Trifluoroethyl)amino]-2-(trifluoromethyl)benzonitrile

To a slurry of 4-amino-2-(trifluoromethyl)benzonitrile (30.09 g, 162 mmol) and NaBH₃CN (21.35 g, 340 mmol) in CH₂CI₂ (160 mL) at ice bath temperature was added neat TFA (160 mL, 2.08 mol), dropwise at a rate such that the internal temperature remained below 5° C. (CAUTION: exothermic reaction with hydrogen gas evolution). Trifluoroacetaldehyde hydrate (52.2 g, 405 mmol) was then added over 5 minutes (CAUTION: slightly exothermic reaction, with gas evolution). After 41 h, the mixture was slowly poured into satd NaHCO₃ (1 L) at 0° C. The mixture was then completely neutralized by portionwise addition of solid NaHCO₃. The mixture was stirred 30 min and precipitated solids were collected by filtration. Organic and aqueous phases of the filtrate were separated, and the aqueous layer extracted with CH₂CI₂ (3 ×150 mL). Combined organic extracts were concentrated to dryness, combined with the solids collected previously, dissolved in EtOAc, washed (H₂O, brine), dried over Na₂SO₄, filtered through a short pad of Celite, and concentrated to dryness. The residue was filtered through a pad of silica gel (EtOAc/hexanes) and concentrated to dryness. Recrystallization from EtOAc/hexanes yielded 32.61 g of the title compound as slightly tan crystalline plates, mp 132.5-134° C: ¹H NMR (300 MHz, MeOH-d₄) δ7.59 (d, J=8.8 Hz, 1H), 7.05 (d, J=2.2 Hz, 1H), 6.92 (dd, J=8.7, 2.4 Hz, 1H), 3.92 (q, J=9.2 Hz, 2H).

### EXAMPLE 115

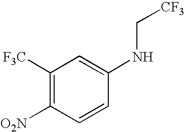 4-Nitro-N-(2,2,2-trifluoroethyl)-3-(trifluoromethyl)aniline

Synthesized as described in example 114 from 4-nitro-3-(trifluoromethyl)aniline: ¹H NMR (300 MHz, CDCI₃) δ8.02 (d, J=9.0 Hz, 1 H), 7.03 (d, J=2.6 Hz 1H), 6.85 (dd, J=9.0, 2.6 Hz, 1 H), 4.82 (bt, J=6.9 Hz, 1 H), 3.91 (overlapping qd, 2H).

### EXAMPLE 116

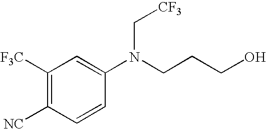 4-[(3-Hydroxypropyl)(2,2,2-trifluoroethyl)amino]-2-(trifluoromethyl)benzonitrile

Synthesized as described in example 89 from 4-[(2,2,2-trifluoroethyl)amino]-2-(trifluoromethyl)benzonitrile and [(3-bromopropyl)oxy](1,1-dimethylethyl)dimethylsilane: MS (ESI) mlz 327 ([M+H]⁺, 32%), 359 ({[M+H]+MeOH}⁺, 69%).

### EXAMPLE 117

4-[(3-Hydroxypropyl)(2,2,2-trifluoroethyl)amino]-3-(trifluoromethyI)benzonitrile 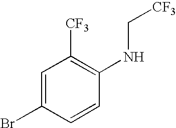
A. 4-Bromo-N-(2,2,2-trifluoroethyl)-2-(trifluoromethyl)aniline

Synthesized as described in example 114 from 4-bromo-2-(trifluoromethyl)aniline: MS (APCI) mlz 322 ([M+H]⁺,⁷⁹Br, 100%), 324 ([M+H]⁺, ⁸¹Br, 94%). 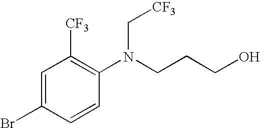
B. 3-[[4-Bromo-24trifluoromethyl)phenyl](2,2,2-trifluoroethyl)amino]-1 -propanol

Synthesized as described in example 89 from example 117A and [(3-bromopropyl)oxy](1,1-dimethylethyl)dimethylsilane: ¹H NMR (300 MHz, CDCI₃) δ7.78 (d, J=2.1 Hz, 1H), 7.65 (dd, J=8.6, 2.1 Hz, 1H), 7.31 (d, J=8.4 Hz, 1H), 3.71-3.56 (m, 4H), 3.30 (t, J=7.4 Hz, 2H), 1.71 (overlapping tt, 2H), 1.35 (bs, 1H). 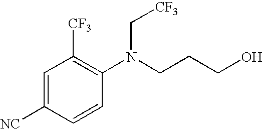
C. 4-[(3-Hydroxypropyl)(2,2,2-trifluoroethyl)amino]-3-(trifluoromethyl)benzonitrile

A mixture of example 117B (0.103 g, 0.21 mmol) and CuCN (0.038 g, 0.42 mmol) in NMP (1 mL) was heated at 125° C. for 4 h. The mixture was cooled, poured into ice water and stirred 30 min. The whole was filtered through Celite, and the filter cake was washed with EtOAc (×2). The aqueous filtrate was extracted with EtOAc, combined organic portions were washed (water, brine), dried over Na₂SO₄, filtered and concentrated in vacuo. The residue was purified by flash chromatography (EtOAc/hexanes), affording 0.022 g of the title compound as a yellow gum: MS (APCI) mlz 327 (M+1).

### EXAMPLE 118

4-Bromo-N,N-bis(2,2,2-trifluoroethyl)-3-(trifluoromethyl)aniline 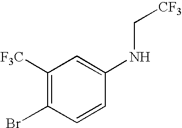
A. 4-Bromo-N-(2,2,2-trifluoroethyl)-3-(trifluoromethyl)aniline

Synthesized as described in example 114 from 4-bromo-3-(trifluoromethyl)aniline: ¹H NMR (300 MHz, CDCI₃) δ7.49 (d, J=8.8 Hz, 1H), 6.98 (d, J=2.9 Hz, 1H), 6.69 (dd, J=8.7, 2.8 Hz, 1H), 4.18 (bt, J=6.9 Hz, 1H), 3.78 (overlapping qd, 2H). 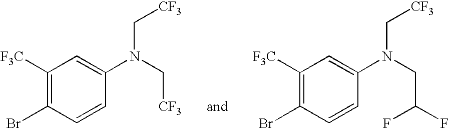
B. 4-Bromo-N,N-bis(2,2,2-trifluoroethyl)-3-(trifluoromethyl)aniline, and 4-Bromo-N-(2,2-difluoroethyl)-N-(2,2,2-trifluoroethyl)3-(trifluoromethyl)anillne

To a solution of 4-bromo-N-(2,2,2-trifluoroethyl)-3-(trifluoromethyl)aniline (0.877 g, 2.72 mmol) in TFA (8 mL) at 0° C. was added NaBH₃CN (0.854 g, 13.6 mmol), portionwise over 5 min. The mixture was stirred 5 min, trifluoroacetaldehyde hydrate (0.70 g, 5.4 mmol) was added. The flask was stoppered with a rubber septum, placed under balloon pressure of nitrogen, and the cooling bath was removed. Excess trifluoroacetaldehyde hydrate (7.0 g; 54,mmol) was added to the stirred mixture via syringe pump over 15 h. The mixture was neutralized by slowly pouring into ice-cold satd NaHCO₃, and the whole was extracted with CH₂CI₂ (×3). Combined organics were washed (water, brine), dried over Na₂SO₄, filtered, and concentrated in vacuo. The residue was purified by flash chromatography (CH₂CI2/hexanes), affording 0.757 g of 4-bromo-N,N-bis(2,2,2-trifluoroethyl)-3-(trifluoromethyl)aniline as a colorless oil that slowly solidified, and 0.99 g of 4-bromo-N-(2,2-difluoroethyl)-N-(2,2,2-trifluoroethyl)-3-(trifluoromethyl)aniline as a colorless oil. 4-Bromo-N,N-bis(2,2,2-trifluoroethyl)-3-(trifluoromethyl)aniline: ¹H NMR (300 MHz, CDCI₃) δ7.58 (d, J=9.0 Hz, 1H), 7.19 (d, J3.1 Hz, 1H), 6.91 (dd, J=8.9, 3.1 Hz, 1H), 4.05 (q, J=8.4 Hz, 4H).

4-Bromo-N-(2,2-difluoroethyl)-N-(2,2,2-trifluoroethyl)-3-(trifluoromethyl)aniline: ¹H NMR (300 MHz, CDCI₃) δ7.56 (d, J=9.1 Hz, 1H), 7.14 (d, J=3.2Hz, 1H), 6.86 (dd, J=8.9, 3.2Hz, 1H), 5.79 (tt, J=55.2,4.0 Hz, 1H), 4.03 (q,J=8.5 Hz, 2H), 3.84 (td, J=13.7, 4.0 Hz, 2H).

### EXAMPLE 119

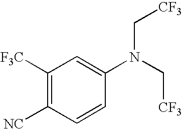 4-[Bis(2,2,2-trifluoroethyl)amino]-2-(trifluoromethyl)benzonitrile

Synthesized as described in example 117C from example 118B: MS (El) mlz 350 (M⁺, 54%), 331 ([M—F]⁺, 31%), 281 ([M—CF₃]⁺, 100%).

### EXAMPLE 120

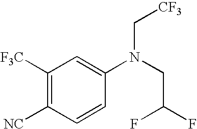 4-[(2,2-Difluoroethyl)(2,2,2-trifluoroethyl)amino]-2-(trifluoromethyl)benzonitrile

Synthesized as described in example 117C from 4-bromo-N-(2,2-difluoroethyl)-N-(2,2,2-trifluoroethyl)-3-(trifluoromethyl)aniline: ¹H NMR (300 MHz, CDCI₃) δ7.71 (d, J =8.8 Hz, 1H), 7.15 (d, J=2.5 Hz, 1H), 7.03 (dd, J=8.9, 2.6 Hz, 1H), 6.03 (t, J=54.8, 3.6Hz, 1H), 4.14 (q, J=8.4 Hz, 2H), 3.94 (td, J=13.9, 3.6Hz, 2H).

### EXAMPLE 121

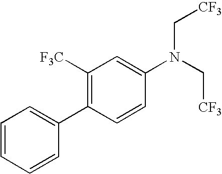 bis(2,2,2-Trifluoroethyl)[2-(trifluoromethyl)-4-biphenylyl]amine

A mixture of example 118B (0.19 g, 0.48 mmol), PhB(OH)₂ (0.064 g, 0.53 mmol), K₂CO₃ (0.165 g, 1.20 mmol), tetrabutylammonium bromide (0.154 g, 0.48 mmol) and Pd(OAc)₂ (0.0011 g, 0.005 mmol) in water (1 mL) was sparged with nitrogen for 10 min, with vigorous stirring, then heated to 70° C. under nitrogen. After 45 min, the mixture was cooled and filtered through Celite. The filter cake was washed with EtOAc (×3) and the aqueous filtrate was extracted with EtOAc (×2). The combined organic portions were washed with brine, dried over Na₂SO₄, filtered and concentrated in vacuo. The residue was purified by flash chromatography (EtOAc/hexanes), affording 0.17 g of the title compound as a colorless gum: MS (EI) mlz 401 (M⁺, 81%), 332 ([M—CF₃]⁺, 100%).

### EXAMPLE 122

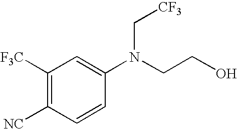 4-[(2-Hydroxyethyl)(2,2,2-trifluoroethyl)amino]-2-(trifluoromethyl)benzonltrile

To a slurry of hexanes-washed NaH (0.162 g of a 60% w/w suspension in mineral oil, 4.0 mmol) in DMF (5 mL) at 0° C. was added a solution of example 118A (0.652 g, 2.02 mmol) in DMF (2 mL), dropwise over 3 minutes. The mixture was stirred 20 min and [(2-bromoethyl)oxy](1,1-dimethylethyl)dimethylsilane (0.86 mL, 4.0 mmol) was added via syringe. The cooling bath was removed and the mixture was stirred at room temp. After 2 hours the mixture was poured into water and the whole was extracted with Et₂O (×3). Combined organic portions were washed (water, brine), dried over Na₂SO₄, filtered and concentrated in vacuo. The residue was purified by flash chromatography (EtOAc/hexanes) affording 0.532 g of a colorless oil.

A mixture of the above oil (0.532 g) and CuCN (0.298 g, 3.33 mmol) in NMP (1 mL) was heated at 140° C. for 4 h. The mixture was cooled, poured into ice water, stirred 20 minutes and the whole was filtered through Celite. The filter cake was washed with EtOAc (×2), and the filtrate was extracted with EtOAc (×2). Combined organic portions were washed (water, brine), dried over Na₂SO₄, filtered, and concentrated in vacuo. The residue was dissolved in EtOH (5 mL), PPTS (0.306 g) was added, and the mixture heated at 80° C. for 3 h. The mixture was cooled, concentrated in vacuo and the residue was partitioned between EtOAc/water. The organic layer was washed (water, brine), dried over Na₂SO₄, filtered and concentrated in vacuo. The residue was purified by flash chromatography (EtOAc/hexanes) affording 0.252 g of the title compound as a colorless solid: ¹H NMR (400 MHz, CDCI₃) δ7.64 (d, J=9.1 Hz,₁H), 7.10 (d, J=2.7 Hz,₁1H), 6.96 (dd, J=8.8, 2.5 Hz,₁H), 4.16 (q, J=8.5 Hz, 2H), 3.95 (overlapping td, 2H), 3.76 (t, J=5.4 Hz, 2H), 1.68 (t, J=4.6 Hz, 1H).

### EXAMPLE 123

4-[[2(Methyloxy)ethyl](2,2,2-trfluoroethyl)amino]-2-(trifluoromethyl)benzonitrile 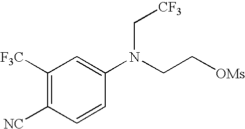
A. 2-[[4-Cyano-3-(trifluoromethyl)phenyl](2,2,2-trifluoroethyl)amino]ethyl methanesulfonate

To a solution of example 122 (0.104 g, 0.333 mmol) in CH₂CI₂ (3 mL) at 0° C. was added Et₃N (0.07 mL, 0.5 mmol), followed by MsCI (0.03 mL, 0.37 mmol). The resulting mixture was stirred 12 h, gradually warming to room temp. The mixture was poured into 10% v/v HCI and the layers were separated. The organic layer was washed (satd NaHCO₃, water, brine), dried over Na₂SO₄, filtered, and concentrated to a colorless gum (0.122 g) which was used without further purification: ¹H NMR (300 MHz, CDCI₃) δ7.71 (d, J=8.8 Hz, 1H), 7.12 (d, J=2.4 Hz, 1H), 6.99 (dd, J=8.7, 2.7 Hz, 1H), 4.43 (t, J=5.7 Hz, 2H), 4.10 (q, J=8.5 Hz, 2H), 3.95 (t, J=5.7 Hz, 2H), 3.02 (s, 3H). 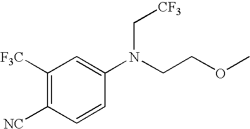
B. 4-[[2-(Methyloxy)ethyl](2,2,2-trifluoroethyl)amino]-2-(trifluoromethyl)benzonitrile

To a solution of example 123A (0.081 g, 0.21 mmol) in MeOH (1 mL) at room temp. was added sodium methoxide (0.056 g, 1.04 mmol) in one portion. The mixture was heated at 50° C. in a sealed vial for 12 h, cooled and poured into 10% HCI, and the whole was extracted with EtOAc (×3). Combined organics were washed (water, brine), dried over Na₂SO₄, filtered and concentrated in vacuo. The residue was purified by preparative RP-HPLC (C18 column, MeCN/water with 0.1% v/v TFA), affording 0.036 g of the title compound as a colorless gum: ¹H NMR (300 MHz, MeOH-d₄) δ7.72 (d, J=8.9 Hz, 1H), 7.27 (d, J=2.5 Hz, 1H), 7.19 (dd, J=8.8, 2.7 Hz, 1H), 4.34 (q, J=9.0 Hz, 2H), 3.79 (t, J=5.2 Hz, 2H), 3.61 (t, J=5.2 Hz, 2H), 3.32 (s, 3H, partially overlapping solvent).

### EXAMPLE 124

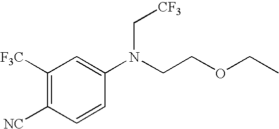 4-[[2-(Ethyloxy)ethyl](2,2,2-trifluoroethyl)amino]-2-(trifluoromethyl)benzonitrile

Synthesized as described in example 123B from example 123A and sodium ethoxide: ¹NMR (300 MHz, MeOH-d₄) δ7.72 (d, J=8.9 Hz,₁H), 7.32 (d, J=2.3 Hz, 1H), 7.19 (dd, J=8.9,2.5Hz, 1H), 4.35 (q, J=8.9Hz, 2H), 3.79 (t, J=5.2Hz, 2H), 3.66 (t, J=5.2 Hz, 2H), 3.47 (q, J=7.0 Hz, 2H), 1.13 (t, J=7.0 Hz, 3H).

### EXAMPLE 125

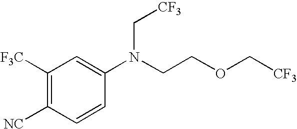 4-((2,2,2-Trifluoroethyl){2-[(2,2,2-trifluoroethyl)oxy]ethyl}amino)-2-(trifluoromethyl)benzonitrile

To a solution of example 122 (0.062 g, 0.20 mmol), 1,1′-(azodicarbonyl)dipiperidine (0.101 g, 0.40 mmol) and 1,1,1-trifluoroethanol (0.14 mL, 2.0 mmol) in dry benzene (4 mL) at room temp was added tri-n-butylphosphine (0.10 mL, 0.40 mmol), dropwise over 2 min. The mixture was stirred 3 h at room temp. and the precipitated solids were removed by filtration. The filtrate was adsorbed onto a small amount of silica gel and purified by flash chromatography (EtOAc/hexanes), affording 0.059 g of the title compound as a pale yellow oil: ¹H NMR (300 MHz, MeOH-d₄) δ7.73 (d, J=8.9 Hz, 1H), 7.31 (d, J=2.4 Hz, 1H), 7.21 (dd, J=8.9, 2.5 Hz, 1H), 4.35 (q, J=8.9 Hz, 2H), 3.92 (q, J=8.9 Hz, 2H), 3.90-3.81 (m, 4H, partially overlapping 3.92).

### EXAMPLE 126

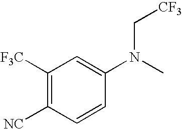 4-[Methyl(2,2,2-trifluoroethyl)aminol-2-(trifluoromethyl)benzonitrile

Synthesized in a manner similar to example 97 from example 114 and iodomethane: MS (ES) mlz283 (M+1).

### EXAMPLE 127

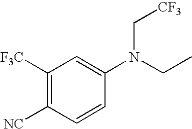 4-[Ethyl(2,2,2-trifluoroethyl)amino]-2-(trifluoromethyl)benzonitrile

Synthesized in a manner similar to example 97 from example 114 and ethyl iodide: MS (ES) mlz297 (M+1).

### EXAMPLE 128

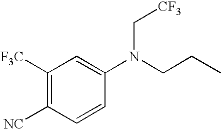 4-[Propyl(2,2,2-trifluoroethyl)amino]-2-(trifluoromethyl)benzonitrile

Synthesized as described in example 97 from example 114 and 1-iodopropane: ¹H NMR (300 MHz, CDCI₃) δ7.73 (d, J=8.7 Hz, 1H), 7.19-7.09 (m, 2H), 4.27 (q, J=8.8 Hz, 2H), 3.51 (t, J=7.9 Hz, 2H), 1.66 (overlapping qt, 2H), 0.96 (t, J=7.3 Hz, 3H).

### EXAMPLE 129

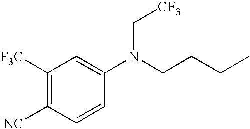 4-[Butyl(2,2,2-trifluoroethyl)amino]-2-(trifluoromethyl)benzonitrile

Step 1. The intermediate 4-[but-2-enyl(2,2,2-trifluoroethyl)amino]-2-(trifluoromethyl)benzonitrile was synthesized (as a mixture of cis/trans olefins) as described in example 97 using crotyl bromide.

Step 2. To a suspension of 5% Pd/C (0.030 g) in EtOAc (3 mL) that was pre-stirred under a hydrogen atmosphere (balloon pressure) for 10 min was added a solution of the above intermediate (0.068 g, 0.21 mmol), in EtOAc (2 mL) and hydrogenated for 2.5 h. The catalyst was filtered off, washed with EtOAc and the filtrate concentrated in vacuo. The residue was purified by silica gel chromatography (2-40% EtOAc- hexane gradient) and the product crystallized from CH₂CI₂-hexane to give the title compound as a white solid (0.053 g, 78% yield): ¹H NMR (400 MHz, CDCI₃) δ7.62 (d, J=8.8 Hz, 1H), 7.01 (d, J=2.6 Hz, 1 H), 6.88 (dd, J=8.8, 2.6 Hz, 1H), 3.95 (q, J =8.6 Hz, 2H), 3.48 (t, J=8.0 Hz, 2H), 1.60 (m, 2H), 1.39 (m, 2H), 0.98 (t, J=7.4 Hz, 3H); MS (ES) mlz 325 (M+1).

### EXAMPLE 130

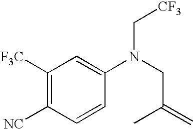 4-[(2-Methylprop-2-enyl)(2,2,2-trifluoroethyl)amino]-2-(trifluoromethyl)benzonitrile

Synthesized as described in example 97 from example 114 and 3-bromo-2-methylprop-1-ene: MS (EI) mlz 323 (M+1).

### EXAMPLE 131

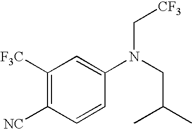 4-[Isobutyl(2,2,2-trifluoroethyl)amino]-2-(trifluoromethyl)benzonitrile

A mixture of example 130 (0.035 g, 0.108 mmol) and 10% Pd/C (0.030 g,50% water) in EtOAc (3 mL) was hydrogenated under balloon pressure for 30 min. The catalyst was filtered off, washed with EtOAc and the filtrate concentrated in vacuo. As judged ¹H NMR, the reaction was incomplete and the residue was subjected to the same reaction conditions for 1 h and worked up similarly. The residue was purified by silica gel chromatography (2-40% EtOAc-hexane gradient) to give the title compound (0.021 g,40% yield): ¹H NMR (400 MHz, CDCI₃) δ7.63 (d, J=8.8 Hz, 1H), 7.04 (d, J=2.6 Hz, 1H), 6.90 (dd, J=8.8, 2.6 Hz, 1H), 4.01 (q, J=8.4 Hz, 2H), 3.33 (d, J=7.5 Hz, 2H), 2.10 (m, 1H), 0.96 (d, J=6.6 Hz, 6H); MS (ES) mlz325 (M+1).

### EXAMPLE 132

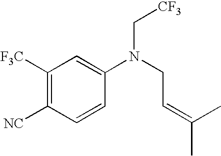 4-[(3-Methylbut-2-enyl)(2,2,2-trifluoroethyl)amino]-2-(trifluoromethyl)benzonitrile

Synthesized as described in example 97 using example 114 and 1-bromo-3-methylbut-2-ene: ¹H NMR (400 MHz, CDCI₃) δ7.62 (d, J=8.8 Hz, 1H), 7.03 (d, J=2.6 Hz, 1H), 6.89 (dd, J=8.8, 2.6 Hz, 1H), 5.07 (t, J=6.4 Hz, 1H), 4.08 (d, J=6.4 Hz, 2H), 3.93 (q, J=8.6 Hz, 2H), 1.76 (s, 6H); MS (ES) mlz 337 (M+1).

### EXAMPLE 133

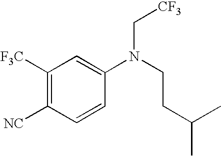 4-[Isopentyl (2,2 ,2-triflu oroethyl) amnino] -2 -(trifu oromethyi) be nzon itri le P Synthesized in a manner similar to example 131 from example 132: ¹H NMR (400 MHz, CDCI₃) δ7.63 (d, J=8.8 Hz, 1H), 7.02 (d, J=2.6 Hz, 1H), 6.87 (dd, J=8.8, 2.6 Hz, 1H), 3.94 (q, J=8.6 Hz, 2H), 3.49 (t, J=8.2 Hz, 2H), 1.64 (m, 1H), 1.51 (m, 2H), 0.98 (d, J=6.6 Hz, 6H); MS (ES) mlz 339 (M+1).

### EXAMPLE 134

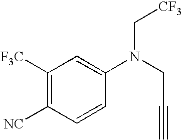 4-[Prop-2-ynyl(2,2,2-trifluoroethyl)aminol-2-(trifluoromethyl)benzonitrile

Synthesized as described in example 97 using example 114 and 3-bromopropyne: ¹H NMR (400 MHz, CDCI₃) δ7.71 (d, J=8.8 Hz, 1H), 7.20 (d, J=2.6 Hz, 1H), 7.06 (dd, J=8.8, 2.6 Hz, 1H), 4.20 (d, J=2.3 Hz, 2H), 4.0 (q, J=8.6 Hz, 2H), 2.35 (t, J=2.3 Hz, 1 H); MS (ES) mlz 307 (M+1).

### EXAMPLE 135

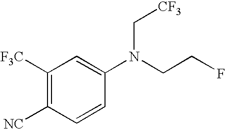 4-1(2-Fluoroethyl)(2,2,2-trffluoroethyl)amino]-2-(trifluoromethyl)benzon0trile

A mixture of example 114 (0.050 g, 0.186 mmol), cesium carbonate (0.121 g, 0.372 mmol) and 1-bromo-2-fluoroethane (0.236 g, 1.86 mmol) in MeCN (3 mL) was heated in a pressure tube at 120° C. under nitrogen for 12 h. Upon cooling, the reaction mixture was filtered, the solids were washed with ethyl acetate, and the filtrate was concentrated under vacuo. The residue was purified by silica gel chromatography (10-60% dichloromethane-hexane gradient) and the product crystallized from hexanes to give the title compound as a white solid (0.029 g, 50% yield): ¹H NMR (400 MHz, CDCI₃) δ7.67 (d, J=8.8 Hz, 1H), 7.07 (d, J=2.6 Hz, 1H), 6.95 (dd, J=8.8, 2.6 Hz, 1H), 4.69 (dt, J=46.9, 4.8 Hz, 2H), 4.10 (q, J=8.6 Hz, 2H), 3.89 (dt, J=24.7, 4.8 Hz, 2H).

### EXAMPLE 136

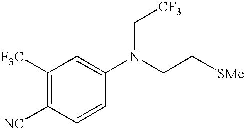 4-[12-(Methylthio)ethyl](2,2,2-trifluoroethyl)amino]-2-(trifluoromethyl)benzonitrile

To a solution of example 123A (0.050 g, 0.128 mmol) in DMF (2 mL), under nitrogen was added sodium thiomethoxide (0.010 g,0.143 mmol). After stirring at room temperature for 20 min, additional sodium thiomethoxide (0.010 g, 0.143 mmol) was added and stirred another 15 min. The mixture was partitioned between Et₂O and water. The organic phase was washed with water and brine, dried (Na₂SO₄) and concentrated in vacuo. The residue was purified by silica gel chromatography (5-40% EtOAc-hexane gradient) and the product was crystallized from CH₂CI₂hexanes to give the title compound (0.075 g, 86% yield): MS (ES) mlz 343 (M+1).

### EXAMPLE 137

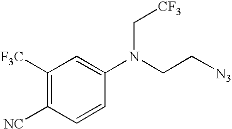 4-[(2-Azidoethyl)(2,2,2-trifluoroethyl)amino]-2-(trifluoromethyl)benzonitrile

A mixture of example 123A (0.195 g, 0.50 mmol) and sodium azide (0.065 g, 1.0 mmol) in DMF (5 mL) was heated at 85° C., under nitrogen, for 45 min. Upon cooling, the mixture was partitioned between Et₂O and water. The organic phase was washed with water and brine, dried (Na₂SO₄) and concentrated in vacuo. The residue was purified by silica gel chromatography (5-40% EtOAc-hexane gradient) to give the title compound (0.154 g, 91% yield): ¹H NMR (400 MHz, CDCI₃) δ7.66 (d, J=8.8 Hz, 1H), 7.07 (s, 1H), 6.97 (d, J=8.8Hz, 1H), 4.11 (q, J=8.5Hz, 2H), 3.72 (t, J=5.8 Hz, 2H), 3.62 (t, J=5.8 Hz, 2H).

### EXAMPLE 138

N-{2-[[4-Cyano-3-(trifluoromethyl)phenyl](2,2,2-trifluoroethyl)amino]ethy}acetamide 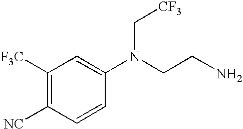
A. 4-[(2-Aminoethyl)(2,2,2-trifluoroethyl)amino]-2-(trifluoromethyl)benzonitrile

A mixture of example 137 (0.150g, 0.445 mmol) and 10% Pd/C (0.120 g) in MeOH (8 mL) was hydrogenated under balloon pressure for 1 h. The catalyst was filtered off and washed with CHCI₃ and MeOH. The filtrate was concentrated in vacuo to give the title compound (0.136 g, 98% crude yield), which was used as such without further purification. 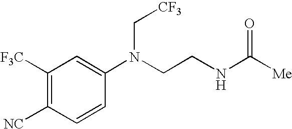
B. N-{2-[[4-Cyano-3-(trifluoromethyl)phenyl](2,2,2-trifluoroethyl)amino]ethyl)acetamide

A solution of example 138A (0.046 g, 0.147 mmol) and Et₃N (0.022 g, 0.22 mmol) in THF (2 mL) was cooled in an ice bath and treated with acetic anhydride (0.017 g, 0.16 mmol). After 10 min, the ice bath was removed and the mixture stirred at room temperature for 30 min. The reaction mixture was partitioned between EtOAc and 0.1 N HCI. The organic phase was washed with 0.1 N HCI and brine, dried (Na₂SO₄), and concentrated in vacuo. The residue was purified by silica gel chromatography (1-6% MeOH-dichloromethane gradient) and the product crystallized from dichloromethane-hexanes to give the title compound as a white solid (0.030 mg, 57% yield for two steps). ¹HNMR (400 MHz, CDCI₃) δ7.64 (d, J=8.8 Hz, 1 H), 7.22 (d, J =2.5 Hz,1H), 7.12 (dd, J=8.8, 2.5 Hz,1H), 5.94 (bs, NH), 4.01 (q, J=8.5 Hz, 2H), 3.67 (t, J=7.1 Hz, 2H), 3.44 (q, J=7.5 Hz, 2H), 1.97 (s, 3H); MS (ES) mlz 354 (M+1).

### EXAMPLE 139

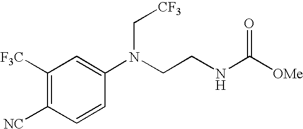 Methyl 2-[[4-cyano-3-(trifluoromethyl)phenyl](2,2,2-trifluoroethyl)amino]ethylcarbamate

Prepared in a manner similar to example 138B from methyl chloroformate: MS (ES) mlz 370 (M+1).

### EXAMPLE 140

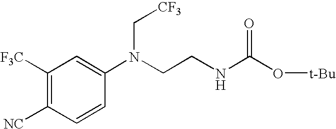 tert-Butyl 2-[[4-cyano-3-(trifluoromethyl)phenyl](2,2,2-trifluoroethyl)amino]ethylcarbamate

Prepared in a manner similar to example 138B from di-tert-butyl dicarbonate: ¹H NMR (400 MHz, CDCI₃) δ7.65 (d, J=8.8 Hz, 1H), 7.16 (s,1H), 7.07 (d, J=8.8 Hz, 1H), 4.75 (bs, NH), 3.98 (q, J=8.6 Hz, 2H), 3.65 (t, J=6.8 Hz, 2H), 3.33 (q, J=6.8 Hz, 2H), 1.39 (s, 9H); MS (ES) mlz412 (M+1).

### EXAMPLE 141

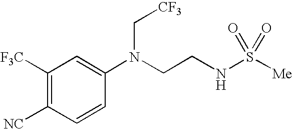 N-(2-[[4-Cyano-3-(trifluoromethyl)phenyl](2,2,2-trifluoroethyl)amino]ethyl}methanesulfonamide

Prepared in a manner similar to example 138B from methanesulfonyl chloride: ¹H NMR (400 MHz, CDCI₃) δ7.68 (d, J=8.8 Hz, 1 H), 7.13 (d, J=2.2 Hz,1H), 7.01 (dd, J=8.8, 2.2 Hz, 1H), 4.49 (t, J=6.1 Hz, NH), 4.05 (q, J=8.4 Hz, 2H), 3.78 (t, J=7.0 Hz, 2H), 3.35 (q, J=7.0 Hz, 2H), 2.99 (s, 3H).

### EXAMPLE 142

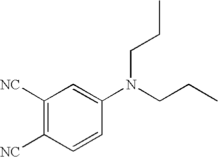 4-(Dipropylamino)phthalonitrile

A mixture of 4-nitrophthalonitrile (0.100 g, 0.58 mmol) and dipropylamine (0.234 g, 2.32 mmol) in DMSO (1.5 mL) was heated under nitrogen in a microwave at 140° C. for 20 min. After cooling, the mixture was partitioned between EtOAc and water. The organic phase was washed with water and brine, dried (Na₂SO₄) and concentrated in vacuo. The residue was purified by silica gel chromatography (5-80% EtOAc-hexanes gradient) and the product crystallized from CH₂CI₂-hexanes to give the title compound as a white solid (0.015 g, 11% yield): MS (ES) mlz 228 (M+1).

### Biological Section

Compounds of the current invention are or are believed to be modulators of the androgen receptor, glucocorticoid receptor, the mineralocorticoid receptor, and/or the progesterone receptor. Activity mediated through these oxosteroid nuclear receptors was measured using the following in vitro and in vivo assays.
In Vitro Assays:
The following abbreviations and sources of materials are used
Fluormone PL Red—a commercially available PR fluoroprobe (PanVera Corp, Product No P2965)
Fluormone GS Red—a commercially available GR fluoroprobe (PanVera Corp, Product No P2894)
Fluormone AL Green—a commercially available AR fluoroprobe (PanVera Corp, Product No P3010)
PR-LBD—Purified human progesterone ligand binding domain tagged with
Glutathione Transferase (PanVera Corp, Product No P2900)
GR—purified human glucocorticoid receptor (PanVera Corp, Product No P2812)
AR-LBD—Purified rat androgen ligand binding domain tagged with Glutathione Transferase (PanVera Corp, Product No P3009)
PR Screening Buffer—100 mM potassium phosphate (pH 7.4), 100 μG/ml bovine gamma globulin, 15% ethylene glycol, 0.02% NaN₃, 10% glycerol (PanVera Corp Product No P2967) with 0.1% w/v CHAPS
AR Screening Buffer—pH 7.5 containing protein stabilizing agents and glycerol (PanVera Corp Product No P3011)
GR Screening Buffer—100 mM potassium phosphate (pH 7.4), 200 mM Na₂MoO₂, 1 mM EDTA, 20% DMSO (PanVera Corp Product No P2814) with GR stabilizing peptide (100 μM) (PanVera Corp Product No P2815)
DTT—dithiothreitol (PanVera Corp Product No P2325)
Discovery Analyst—is an FP reader
DMSO—dimethylsulphoxide
   Progesterone Receptor Fluorescence Polarization Assay:

The progesterone receptor fluorescence polarization assay is used to investigate the interaction of the compounds with the progesterone receptor.

Compounds are added to the 384 well black plates to a final volume of 0.5 μl. Sufficient Fluormone PL Red and PR-LBD are defrosted on ice to give a final concentration of 2 nM and 40 nM, respectively. PR screening buffer is chilled to 4° C. prior to addition of DTT to give a final concentration of 1 mM. The Fluormone PL Red and PR-LBD in PR Screening Buffer are added to compound plates to give a final volume of 10 μL. The assay is allowed to incubate at 20-22° C. for 2 hours. The plates are counted in a Discovery Analyst with suitable 535 nM excitation and 590 nM emission interference filters. Compounds that interact with the PR receptor result in a lower fluorescence polarization reading. Test compounds are dissolved and diluted in DMSO. Compounds are assayed in singlicate, a four parameter curve fit of the following form being applied

$$y = \frac{a - d}{1 + {( \frac{x}{c} )}^{b}} + d$$

where a is the minimum, b is the Hill slope, c is the IC₅₀ and d is the maximum. Maximum and minimum values are compared to adhesion in the absence of compound and in the presence of 10⁻⁵ M progesterone. Data is presented as the mean pIC₅₀ with the standard error of the mean of n experiments. Compounds with plC₅₀ greater than 5.0 and a % max greater than 50 are considered desirable.
Androgen Receptor Fluorescence Polarization Assay:

The androgen receptor fluorescence polarization assay is used to investigate the interaction of the compounds with the androgen receptor.

Compounds are added to the 384 well black plates to a final volume of 0.5 μl. Sufficient Fluormone AL Green and AR-LBD are defrosted on ice to give a final concentration of 1 nM and 25 nM, respectively. AR screening buffer is chilled to 4° C. prior to addition of DTT to give a final concentration of 1 mM. The Fluormone AL Green and AR-LBD in AR Screening Buffer are added to compound plates to give a final volume of 10 μL. The assay is allowed to incubate at 20° C. for 5 hours. The plates are counted in a Discovery Analyst with suitable 485 nM excitation and 535 nM emission interference filters. Compounds that interact with the AR receptor result in a lower fluorescence polarization reading. Test compounds are dissolved and diluted in DMSO. Compounds are assayed in singlicate, a four parameter curve fit of the following form being applied

$$y = \frac{a - d}{1 + {( \frac{x}{c} )}^{b}} + d$$

where a is the minimum, b is the Hill slope, c is the IC₅₀ and d is the maximum. Maximum and minimum values are compared to adhesion in the absence of compound and in the presence of 10⁻⁵M dihydrotestosterone. Data is presented as the mean pIC₅₀ with the standard error of the mean of n experiments. Compounds with pIC50greater than 5.0 and a % max greater than 50 are considered desirable.
Glucocorticoid Receptor Fluorescence Polarization Assay

The glucocorticoid receptor fluorescence polarization assay is used to investigate the interaction of the compounds with the glucocorticoid receptor.

Compounds are added to the 384 well black plates to a final volume of 0.5 μl. Sufficient Fluormone GS Red and GR are defrosted on ice to give a final concentration of 1 nM and 4 nM, respectively. GR screening buffer is chilled to 4° C. prior to addition of DTT to give a final concentration of 1 mM. The Fluormone GS Red, and GR in GR Screening Buffer are added to compound plates to give a final volume of 10 μL. The assay is allowed to incubate at 4° C. for 12 hours. The plates are counted in a Discovery Analyst with suitable 535 nM excitation and 590 nM emission interference filters. Compounds that interact with the GR receptor result in a lower fluorescence polarization reading. Test compounds are dissolved and diluted in DMSO. Compounds are assayed in singlicate, a four parameter curve fit of the following form being applied

$$y = \frac{a - d}{1 + {( \frac{x}{c} )}^{b}} + d$$

where a is the minimum, b is the Hill slope, c is the EC₅₀ and d is the maximum. Maximum and minimum values are compared to adhesion in the absence of compound and in the presence of 10⁻⁵M dexamethasone. Data is presented as the mean pIC₅₀ with the standard error of the mean of n experiments. Compounds with plC50greater than 5.0 and a % max greater than 50 are considered desirable.
Transient Transfection Assay:

Cotransfection assays using full-length hAR were performed in CV-1 cells (monkey kidney fibroblasts). The cells were seeded in charcoal-stripped medium in 96-well plates (24,000 cells/well) and incubated overnight. Transient transfections were carried out using the following plasmids: pSG5-AR, MMTV LUC reporter, β-actin SPAP, and pBluescript (filler DNA). The cell plates were then incubated for 6-20 hours. The transfection mixture was washed away and then the cells were drugged with doses ranging from 10⁻¹⁰ to 10⁻⁵. Two replicates were used for each sample. Incubation with drug was continued for 14 hours. A spectrophotometer was used for SPAP measurements, while a topcounter was used to read the results from the luciferase assay. The ratio of luciferase activity to SPAP activity was calculated to normalize the variance in cell number and transfection efficiency.

Data analysis:

Data were reduced using RoboFit99. The results were expressed as percent of maximum as calculated by the following formulas:

$$fold activation = \frac{\begin{matrix}( ( \frac{( Luc )}{( SPAP - SPAP substrate blank avg . )} ) - \\ basal activation )\end{matrix}}{basal {activation}^{*}}$$

^{*}basal activation per plate=(Luc vehicle)/(SPAP vehicle−substrate blank average)
% max. =(^{fold activaton of unknown}/_{positive control fold activation avg.})×100

Curves were fit from these data using RoboFit to determine EC₅₀'s for agonists and IC₅₀'s for antagonists using the following equation:
Y=((Vmax^{*}x)/(K+x))+Y2

These values were converted to pEC₅₀'s and plC₅₀'s for posting by using the following equations:
pEC₅₀=−log(EC₅₀)
pIC₅₀=−log(IC₅₀)

For antagonist assays, the percent maximum response antagonist was calculated by the following formula in which Yₘᵢₙ and Yₘₐₓ are curve asymptotes at the maximum or minimum concentration tested:
% max. resp. ant.=100^{*}(1−Yₘᵢₙ/Yₘₐₓ)

For antagonist assays, pkb's were calculated using the following formula:
pKb=IC₅₀ of unknown/((1+^{*}conc.^{*})/DHT EC₅₀ average)
where ^{*}conc.^{*}=concentration of DHT used as the agonist in the medium for the antagonist experiment, expressed in nM. This concentration was set at twice pEC₅₀. This would be 0.2 for AR.

Compounds with a pXC₅₀ greater than 5.0 are considered desirable.

Castrated Male Rat Model (ORX Rat)

The activity of the compounds of the present invention as modulators of the androgen receptor was investigated using a castrated male rat model (ORX) as described in C. D. Kockakian, Pharmac. Therap. B 1(2), 149-177 (1975); C. Tobin and Y. Joubert, Developmental Biology 146,131-138 (1991); J. Antonio, J. D. Wilson and F. W. George, J Appl. PhysioL 87(6) 2016-2019 (1999)) the disclosures of which herein are included by reference.

It has been well defined that androgens play important roles in the maintenance and growth of many tissues in both animals and humans. Muscles, like the levator ani and bulbocavernosus, and sexual accessory organs, such as the prostate glands and seminal vesicles have high expression levels of the androgen receptor and are known to respond quickly to exogenous androgen addition or androgen deprivation through testicular ablation. Castration produces dramatic atrophy of muscle and sexual accessory organs; whereas the administration of exogenous androgens to the castrated animal results in effective hypertrophy of these muscles and sexual accessory organs. Although the levator ani muscle, also known as the dorsal bulbocavernosus, is not ‘true skeletal muscle’ and definitely sex- linked, it is reasonable to use this muscle to screen muscle anabolic activities of test compounds because of its androgen responsiveness, and simplicity of removal.

Male Sprague-Dawley rats weighing 160-180 grams were used in the assay. The rats were singly caged upon receiving and throughout the study. Bilateral orchidectomies were performed in sterilized surgical conditions under isoflurane anesthesia. An anteroposterior incision was made in the scrotum. The testicles were exteriorized and the spermatic artery and vas deferens were ligated with 4.0 silk 0.5 cm proximal to the ligation site. The testicles then were removed by a surgical scissors distal to the ligation sites. The tissue stumps were returned to the scrotum, the scrotum and overlying skin were closed by a surgical stapler. The Sham-ORX rats underwent all procedures except ligation and scissors cutting. The rats were assigned randomly into study groups 7-10 days post surgery based on the body weight.

Dihydrotestosterone (DHT) was used as a positive control (1-10 mg/kg s.c.). Compounds of the current invention were administered subcutaneously or orally for 4-28 days. The rats were weighed daily and doses were adjusted accordingly. The general well being of the animal was monitored throughout the course of the study.

At the end of the study, the rats were euthanized in a CO₂ chamber. The ventral prostate glands(VP), seminal vesicles(SV), levator ani muscle(LA) and bulbocavernosus(BC) were carefully dissected. The tissues were blotted dry, the weights were recorded, and then saved for histological and molecular analysis. The VP and SV weights serve as androgenic indicators and LA and BC are anabolic indicators. The ratio of anabolic to androgenic activities was used to evaluate the test compounds. Serum luteinizing hormone(LH), follicle stimulating hormone(FSH) and other potential serum markers of anabolic activities were also analyzed.

In general, desirable compounds show levator ani hypertrophy and very little prostate stimulation

Test compounds were employed in free or salt form.

All research complied with the principles of laboratory animal care (NIH publication No. 85-23, revised 1985) and GlaxoSmithKline policy on animal use.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included within the scope of the appended claims.

What is claimed is:

1. A compound of formula (I):
including salts, solvates, and physiologically functional derivatives thereof, wherein
R¹ is H, cyano, nitro, halogen, haloalkyl, alkyl, alkenyl, alkynyl, hydroxy, alkoxy, haloalkoxy, —OC(O)R⁸, or aryl;
R² is H, cyano, nitro, halogen, haloalkyl, alkyl, alkenyl, alkynyl, hydroxy, alkoxy, haloalkoxy, —OC(O)R⁸, or aryl;
R³ is cyano, nitro, halogen, haloalkyl, heterocyclyl, hydroxy, alkoxy, haloalkoxy, —OC(O)R⁸, —C(O)₂R⁸, —CONHR⁸, —C(O)R⁸, —S(O)ₙR⁸, —SO₂N(R⁸)₂, —NHC(O)R⁸, or —NHSO₂R⁸;
R⁴ is H, cyano, nitro, halogen, haloalkyl, alkyl, alkenyl, alkynyl, hydroxy, alkoxy, haloalkoxy, —OC(O)R , or aryl;
R⁵ is H, cyano, nitro, halogen, haloalkyl, alkyl, alkenyl, alkynyl, hydroxy, alkoxy, haloalkoxy, —OC(O)R⁸, or aryl;
where at least one of R¹, R², R⁴, and R⁵ is not H;
each of R⁶ and R⁷ independently are selected from H or —(Ra)ₓ-R⁹;
R^{a} is a C₁-C₈ alkylene chain, where x is 0 or 1;
each R8 independently is H, alkyl, alkenyl, alkynyl, cycloalkyl, heterocyclyl, aryl, aralkyl, heteroaryl, or heteroaralkyl; R⁹ is alkyl, haloalkyl, hydroxyalkyl, alkenyl, alkynyl, alkoxy, alkylthio, haloalkoxy, cycloalkyl, formyl, azido, or —NR¹⁰R¹¹;
R¹⁰ and R¹¹ each independently are H, alkyl, —C(O)H, —C(O)R¹², —C(O)OR¹², or —SO₂R¹²; and R¹² is alkyl.

2. The compound of claim 1 wherein R¹, R⁵, or both are H.

3. The compound of claim 1 wherein R², R⁴, or both are H.

4. The compound of claim 1 wherein alkyl is C₁-C₆ alkyl.

5. The compound of claim 4 wherein alkyl is C₁-C₃ alkyl

6. The compound of claim 1 wherein alkoxy is C₁-C₆ alkoxy.

7. The compound of claim 6 wherein alkoxy is C₁-C₂ alkoxy.

8. The compound of claim 1 wherein haloalkyl is C₁-C₆ haloalkyl.

9. The compound of claim 8 wherein haloalkyl is trifluoromethyl or trifluoroethyl.

10. The compound of claim 1 wherein alkenyl is C₂-C₆ alkenyl.

11. The compound of claim 10 wherein alkenyl is isopropenyl, isobutenyl, or allyl.

12. The compound of claim 1 wherein alkynyl is C₂-C₆ alkynyl.

13. The compound of claim 12 wherein alkynyl is propynyl.

14. The compound of claim 1 wherein cycloalkyl is C₃-C₆ cycloalkyl.

15. The compound of claim 14 wherein cycloalkyl is cyclopropyl, cyclopentyl, or cyclohexyl.

16. The compound of claim 1 wherein R¹or R⁵is nitro, alkyl, haloalkyl, or halogen.

17. The compound of claim 1 wherein R² or R⁴is nitro, cyano, alkyl, haloalkyl, halogen, or hydroxy.

18. The compound of claim 1 wherein R³ is cyano, nitro, or halogen.

19. The compound of claim 18 wherein one or more of R¹, R² R⁴, or R⁵ is haloalkyl.

20. The compound of claim 19 wherein R² or R⁴is haloalkyl.

21. The compound of claim 18 wherein one or more of R¹, R², R⁴, or R⁵ is halogen.

22. The compound of claim 21 wherein R² or R ⁴is halogen.

23. The compound of claim 22 wherein halogen is chloro.

24. The compound of claim 18 wherein when R³ is cyano, one or more of R¹, R², R⁴, or R⁵ is nitro.

25. The compound of claim 18 wherein when R³ is nitro, one or more of R¹, R², R⁴,or R⁵ is cyano.

26. The compound of claim 18 wherein R³ is cyano and one of R² or R⁴ is cyano.

27. The compound of claim 1 wherein R^{a} is a substituted alkylene, substituted with one or more of alkyl, alkenyl, or hydroxyl.

28. The compound of claim 27 wherein R^{a} is methylene.

29. A compound selected from:
4-[(Cyclopropylmethyl)(propyl)amino]-2-(trifluoromethyl)benzonitrile
4-[(cyclopropylmethyl)(propyl)amino]-2-nitrobenzonitrile
4-(diallylamino)-2-(trifluoromethyl)benzonitrile
5-[allyl(cyclopentyl)amino]-2-nitrobenzonitrile
4-[butyl(propyl)amino]-2-nitrobenzonitrile
4-[ethyl(2-methyl-2-propenyl)amino]-2-nitrobenzonitrile
4-[butyl(ethyl)amino]-2-(trifluoromethyl)benzonitrile
4-(dipropylamino)-2-(trifluoromethyl)benzonitrile
N-butyl-N-ethyl-3-methyl-4-nitroaniline
5-(diallylamino)-2-nitrobenzonitrile
5-[(cyclopropylmethyl)(propyl)amino]-2-nitrobenzonitrile
4-(diallylamino)-2-nitrobenzonitrile
3-methyl-4-nitro-N,N-dipropylaniline
4-[sec-butyl(propyl)amino]-2-nitrobenzonitrile
5-[butyl(ethyl)amino]-2-nitrobenzonitrile
2-chloro-4-[(cyclopropylmethyl)(propyl)amino]benzonitrile
5-[butyl(propyl)amino]-2-nitrobenzonitrile
5-[(2-methoxyethyl)(methyl)amino]-2-nitrobenzonitrile
2-chloro-4-(diallylamino)benzonitrile
4-[(2-methoxyethyl)(propyl)amino]-2-nitrobenzonitrile
4-[allyl(cyclopentyl)amino]-2-nitrobenzonitrile
4-[ethyl(2-methyl-2-propenyl)amino]-2-(trifluoromethyl)benzonitrile
2-chloro-4-[[2-(dimethylamino)ethyl](methyl)amino]benzonitrile
N-(2-methoxyethyl)-N,2-dimethyl-4-nitroaniline
N-allyl-N-cyclopentyl-4-nitro-3-(trifluoromethyl)aniline
5-(dipropylamino)-2-nitrophenol
2-chloro-4-(dipropylamino)benzonitrile
5-[ethyl(2-methyl-2-propenyl)amino]-2-nitrobenzonitrile
N,N-diallyl-4-nitro-3-(trifluoromethyl)aniline
N,N-diallyl-2-methyl-4-nitroaniline
5-[(cyclopropylmethyl)(propyl)amino]-2-nitrophenol
N-(2-methoxyethyl)-3-methyl-4-nitro-N-propylaniline
4-[butyl(propyl)amino]-2-chlorobenzonitrile
N-butyl-2-chloro-N-methyl-4-nitroaniline
4-(dipropylamino)-2-nitrobenzonitrile
2-chloro-4-[(2-methoxyethyl)(propyl)amino]benzonitrile
N,N-diallyl-3-methyl-4-nitroaniline
N-(sec-butyl)-4-nitro-N-propyl-3-(trifluoromethyl)aniline
4-(dipentylamino)-2-nitrobenzonitrile
N-(cyclopropylmethyl)-3-methyl-4-nitro-N-propylaniline
5-(dipropylamino)-2-nitrobenzonitrile
N,N-dibutyl-4-nitro-3-(trifluoromethyl)aniline
N1-(2-chloro4-nitrophenyl)-N1,N2,N2-trimethyl-1,2-ethanediamine
5-[sec-butyl(propyl)amino]-2-nitrobenzonitrile
N-ethyl-N-(2-methyl-2-propenyl)-4-nitro-3-(trifluoromethyl)aniline
N-(2-methoxyethyl)-4-nitro-N-propyl-3-(trifluoromethyl)aniline
4-[butyl(ethyl)amino]-2-chlorobenzonitrile
5-(dibutylamino)-2-nitrobenzonitrile
4-[butyl(ethyl)amino]-2-nitrobenzonitrile
4-[(2-methoxyethyl)(propyl)amino]-2-(trifluoromethyl)benzonitrile
5-[(2-methoxyethyl)(propyl)amino]-2-nitrobenzonitrile
4-[allyl(cyclopentyl)amino]-2-(trifluoromethyl)benzonitrile
4-[butyl(propyl)amino]-2-(trifluoromethyl)benzonitrile
N,N-dibutyl-3-methyl4-nitroaniline
4-[methyl(octyl)amino]-2-nitrobenzonitrile
4-(dibutylamino)-2-(trifluoromethyl)benzonitrile
N-(cyclopropylmethyl)4-nitro-N-propyl-2-(trifluoromethyl)aniline
N-allyl-N-cyclohexyl-4-nitro-3-(trifluoromethyl)aniline
4-[(2-methoxyethyl)(methyl)amino]-3-(trifluoromethyl)benzonitrile
4-(diallylamino)-3-nitrobenzonitrile
N-1 -(2-chloro-4-nitrophenyl)-N 1, N3, N3-trimethyl-1 ,3-propanediamine
N,N-bis(2-methoxyethyl)-4-nitro-3-(trifluoromethyl)aniline
4-[sec-butyl(propyl)amino]-2-(trifluoromethyl)benzonitrile
2-chloro-4-[ethyl(2-methyl-2-propenyl)amino]benzonitrile
N-cyclohexyl-N-ethyl-4-nitro-3-(trifluoromethyl)aniline
2-chloro-4-(dibutylamino)benzonitrile
4-[cyclohexyl(ethyl)amino]-2-nitrobenzonitrile
4-[bis(2-ethoxyethyl)amino]-3-chlorobenzonitrile
2-chloro-N-(2-methoxyethyl)-N-methyl-4-nitroaniline
N-butyl-N-ethyl-4-nitro-3-(trifluoromethyl)aniline
N-(sec-butyl)-3-methyl-4-nitro-N-propylaniline
N-(2-methoxyethyl)-N-methyl-4-nitro-2-(trifluoromethyl)aniline
4-{bis[3-(dimethylamino)propyl]amino}-2-(trifluoromethyl)benzonitrile
N-(cyclopropylmethyl)-4-nitro-N-propyl-3-(trifluoromethyl)aniline
4-[(methyl)(octyl)amino]-2-(trifluoromethyl)benzonitrile
4-(propylamino)-2-(trifluoromethyl)benzonitrile
4-nitro-N-propyl-3-(trifluoromethyl)aniline
3-{[4-nitro-3-(trifluoromethyl)phenyl]amino}propan-1-ol
4-[(3-hydroxypropyl)amino]-2-(trifluoromethyl)benzonitrile
4-{[2-hydroxy-1-(hydroxymethyl)ethyl]amino}-2-(trifluoromethyl)benzonitrile
4-(dimethylamino)-2-(trifluoromethyl)benzonitrile
4-(diethylamino)-2-(trifluoromethyl)benzonitrile
4-[methyl(2-methylpropyl)amino]-2-(trifluoromethyl)benzonitrile
4-[(cyclopropylmethyl)amino]-2-(trifluoromethyl)benzonitrile
4-[(cyclopropylmethyl)(3-hydroxypropyl)amino]-2-(trifluoromethyl)benzonitrile
4-[(cyclopropylmethyl)(2-hydroxyethyl)amino]-2-(trifluoromethyl)benzonitrile
N-(cyclopropylmethyl)-4-nitro-3-(trifluoromethyl)aniline
3-{(cyclopropylmethyl)[4-nitro-3-(trifluoromethyl)phenyl]amino}-1 -propanol
2-{(cyclopropylmethyl)[4-nitro-3-(trifluoromethyl)phenyl]amino}ethanol
4-[(cyclopropylmethyl)amino]-3-(trifluoromethyl)benzonitrile
1 -[4-[(cyclopropymethyl)(propyl)amino]-2-(trifluoromethyl)phenyl]ethanone
4-[(1 -cyclopropylethyl)amino]-2-(trifluoromethyl)benzonitrile
4-[allyl(1-cyclopropylethyl)amino]-2-(trifluoromethyl)benzonitrile
4-[(2,2-dimethylpropyl)amino]-2-(trifluoromethyl)benzonitrile
4-[(2,2-dimethylpropyl)(3-hydroxypropyl)amino]-2-(trifluoromethyl)benzonitrile
4-[(2,2-dimethylpropyl)(2-propen-1 -yl)amino]-2-(trifluoromethyl)benzonitrile
4-[(2,3-dihydroxypropyl)(2,2-dimethylpropyl)amino]-2-(trifluoromethyl)benzonitrile
4-[(2,2-dimethylpropyl)(2-oxoethyl)amino]-2-(trifluoromethyl)benzonitrile
4-[(2,2-dimethylpropyl)(2-hydroxyethyl)amino]-2-(trifluoromethyl)benzonitrile
4-[(2,2-dimethylpropyl)(propyl)amino]-2-(trifluoromethyl)benzonitrile
4-[(1,1 -dimethylethyl)amino]-2-(trifluoromethyl)benzonitrile
N-(1 , 1 -dimethylethyl)-4-nitro-3-(trifluoromethyl)aniline
N-(cyclopropylmethyl)-N-(1,1-dimethylethyl)-4-nitro-3-(trifluoromethyl)aniline
4-[(1,1-dimethylethyl)(2-propen-1-yl)amino]-2-(trifluoromethyl)benzonitrile
4-[(1,1 -dimethylethyl)(2-oxoethyl)amino]-2-(trifluoromethyl)benzonitrile
4-[(1,1 -dimethylethyl)(propyl)amino]-2-(trifluoromethyl)benzonitrile
4-{(3-hydroxypropyl)[(1S)-1-methylpropyl]amino}-2-(trifluoromethyl)benzonitrile
2-(trifluoromethyl)-4-[(3,3,3-trifluoropropyl)amino]benzonitrile
4-[bis(2-fluoroethyl)amino]-2-(trifluoromethyl)benzonitrile
4-[(2,2,2-trifluoroethyl)amino]-2-(trifluoromethyl)benzonitrile
4-nitro-N-(2,2,2-trifluoroethyl)-3-(trifluoromethyl)aniline
4-[(3-hydroxypropyl)(2,2,2-trifluoroethyl)amino]-2-(trifluoromethyl)benzonitrile
4-[(3-hydroxypropyl)(2,2,2-trifluoroethyl)amino]-3-(trifluoromethyl)benzonitrile
4-bromo-N,N-bis(2,2,2-trifluoroethyl)-3-(trifluoromethyl)aniline
4-[bis(2,2,2-trifluoroethyl)amino]-2-(trifluoromethyl)benzonitrile
4-[(2,2-difluoroethyl)(2,2,2-trifluoroethyl)amino]-2-(trifluoromethyl)benzonitrile
bis(2,2,2-trifluoroethyl)[2-(trifluoromethyl)-4-biphenylyl]amine
4-[(2-hydroxyethyl)(2,2,2-trifluoroethyl)amino]-2-(trifluoromethyl)benzonitrile
4-[[2-(methyloxy)ethyl](2,2,2-trifluoroethyl)amino]-2-(trifluoromethyl)benzonitrile
4-[[2-(ethyloxy)ethyl](2,2,2-trifluoroethyl)amino]-2-(trifluoromethyl)benzonitrile
4-((2,2,2-trifluoroethyl)(2-[(2,2,2-trifluoroethyl)oxy]ethyl~amino)-2-(trifluoromethyl)benzonitrile
4-[methyl(2,2,2-trifluoroethyl)amino]-2-(trifluoromethyl)benzonitrile
4-[ethyl(2,2,2-trifluoroethyl)amino]-2-(trifluoromethyl)benzonitrile
4-[propyl(2,2,2-trifluoroethyl)amino]-2-(trifluoromethyl)benzonitrile
4-[butyl(2,2,2-trifluoroethyl)amino]-2-(trifluoromethyl)benzonitrile
4-[(2-methylprop-2-enyl)(2,2,2-trifluoroethyl)amino]-2-(trifluoromethyl)benzonitrile
4-[isobutyl(2,2,2-trifluoroethyl)amino]-2-(trifluoromethyl)benzonitrile
4-[(3-methylbut-2-enyl)(2,2,2-trifluoroethyl)amino]-2-(trifluoromethyl)benzonitrile
4-[isopentyl(2,2,2-trifluoroethyl)amino]-2-(trifluoromethyl)benzonitrile
4-[prop-2-ynyl(2,2,2-trifluoroethyl)amino]-2-(trifluoromethyl)benzonitrile
4-[(2-fluoroethyl)(2,2,2-trifluoroethyl)amino]-2-(trifluoromethyl)benzonitrile
4-[[2-(methylthio)ethyl](2,2,2-trifluoroethyl)amino]-2-(trifluoromethyl)benzonitrile
4-[(2-azidoethyl)(2,2,2-trifluoroethyl)amino]-2-(trifluoromethyl)benzonitrile
N-{2-[[4-cyano-3-(trifluoromethyl)phenyl](2,2,2-trifluoroethyl)amino]ethyl}acetamide
methyl 2-[[4-cyano-3-(trifluoromethyl)phenyl](2,2,2-trifluoroethyl)amino]ethylcarbamate
tert-butyl 2-[[4-cyano-3-(trifluoromethyl)phenyl](2,2,2-trifluoroethyl)amino]ethylcarbamate
N-{2-[[4-cyano-3-(trifluoromethyl)phenyl](2,2,2-trifluoroethyl)amino]ethyl} methanesulfonamide; and
4-(dipropylamino)phthalonitrile.

30. A compound of formula (I):
including salts, solvates, and physiologically functional derivatives thereof, wherein
R¹ is H, cyano, nitro, halogen, haloalkyl, alkyl, alkenyl, alkynyl, hydroxy, alkoxy, haloalkoxy, —OC(O)R , or aryl;
R² is H, cyano, nitro, halogen, haloalkyl, alkyl, alkenyl, alkynyl, hydroxy, alkoxy, haloalkoxy, —OC(O)R⁸, or aryl;
R³ is cyano, nitro, halogen, haloalkyl, heterocyclyl, hydroxy, alkoxy, haloalkoxy, —OC(O)R⁸, —C(O)₂R⁸, —CONHR⁸, —C(O)R⁸, —S(O)ₙR⁸, —SO₂N(R⁸)₂, —NHC(O)R⁸, or —NHSO₂R⁸;
R⁴ is H, cyano, nitro, halogen, haloalkyl, alkyl, alkenyl, alkynyl, hydroxy, alkoxy, haloalkoxy, —OC(O)R⁸, or aryl;
R⁵ is H, cyano, nitro, halogen, haloalkyl, alkyl, alkenyl, alkynyl, hydroxy, alkoxy, haloalkoxy, —OC(O)R⁸, or aryl;
each of R⁶ and R⁷ independently are selected from H or —(R^{a})ₓ-R⁹;
R^{a} is a C₁-C₈ alkylene chain, where x is 0 or 1;
each R⁸ independently is H, alkyl, alkenyl, alkynyl, cycloalkyl, heterocyclyl, aryl, aralkyl, heteroaryl, or heteroaralkyl;
R⁹ is alkyl, haloalkyl, hydroxyalkyl, alkenyl,-alkynyl, alkoxy, alkylthio, haloalkoxy, cycloalkyl, formyl, azido, or —NR¹⁰R¹¹;
R¹⁰ and R¹¹ each independently are H, alkyl, —C(O)H, —C(O)R¹², —C(O)OR¹² or —SO₂R¹²; and R¹² is alkyl.

31. The compound of claim 30 wherein each of R¹, R², R⁴, and R⁵ are H.

32. The compound of claim 30 wherein R³ is cyano or nitro.

33. The compound of claim 30 wherein the compound is:
N,N-diallyl-4-nitroaniline
N-(cyclopropylmethyl)-4-nitro-N-propylaniline
4-(dipropylamino)benzonitrile; or
4-nitro-N,N-dipropylaniline.

34. (canceled)

35. A pharmaceutical composition comprising a compound according to claim 1, and a pharmaceutically acceptable carrier.

36-41. (canceled)

42. A method for the treatment or prophylaxis of osteoporosis, muscle wasting, frailty, cardiovascular disease, breast cancer, uterine cancer, prostatic hyperplasia, prostate cancer, dyslipidemia, menopausal vasomotor conditions, urinary incontinence, artherosclerosis, libido enhancement, depression, uterine fibroid disease, aortic smooth muscle cell proliferation, endometriosis, or ADAM comprising the administration of a compound according to claim 1.

43. A pharmaceutical composition comprising a compound according to claim 29, and a pharmaceutically acceptable carrier.

44. A pharmaceutical composition comprising a compound according to claim 30, and a pharmaceutically acceptable carrier.

45. A method for the treatment or prophylaxis of osteoporosis, muscle wasting, frailty, cardiovascular disease, breast cancer, uterine cancer, prostatic hyperplasia, prostate cancer, dyslipidemia, menopausal vasomotor conditions, urinary incontinence, artherosclerosis, libido enhancement, depression, uterine fibroid disease, aortic smooth muscle cell proliferation, endometriosis, or ADAM comprising the administration of a compound according to claim 29.

46. A method for the treatment or prophylaxis of osteoporosis, muscle wasting, frailty, cardiovascular disease, breast cancer, uterine cancer, prostatic hyperplasia, prostate cancer, dyslipidemia, menopausal vasomotor conditions, urinary incontinence, artherosclerosis, libido enhancement, depression, uterine fibroid disease, aortic smooth muscle cell proliferation, endometriosis, or ADAM comprising the administration of a compound according to claim 30.
